(19) United States
(12) Patent Application Publication
(10) Pub. No.: US 2026/0327197 A1
(43) Pub. Date: Oct. 22, 2026

(54) METHODS AND COMPOSITIONS FOR OXIDIZED HYDROCARBON PRODUCTION

(71) Applicants: Brookhaven Science Associates, LLC, Upton, NY (US); Universidad Nacional del Litoral, Santa Fe (AR)

(72) Inventors: Darío José Stacchiola, South Setauket, NY (US); Esteban L. Fornero, Sauce Viejo (AR); Adrian L. Bonivardi, Santa Fe (AR); Marta V. Bosco, San Jose del Rincón (AR); Jorge Anibal Boscoboinik, Stony Brook, NY (US)

(21) Appl. No.: 19/468,267

(22) Filed: Feb. 3, 2026

Related U.S. Application Data
(60) Provisional application No. 63/752,942, filed on Feb. 3, 2025.

(51) Int. Cl. C07D 301/08; B01J 23/825

(52) U.S. Cl. CPC C07D 301/08; B01J 23/825

(57) ABSTRACT

In accordance with the purpose(s) of the present disclosure, as embodied and broadly described herein, the disclosure, in one aspect, relates to methods and compositions for production of an oxidized hydrocarbon. In one aspect, the method comprises flowing a first gas mixture over a catalyst, wherein the first gas mixture comprises a reducing agent; and flowing a second gas mixture comprising a hydrocarbon over the catalyst, thereby producing a gaseous product comprising an oxidized hydrocarbon; wherein the catalyst comprises Cuⁿ⁺ ions dispersed in an oxide matrix, where n is an integer ranging from 0 to 2; and wherein the oxide matrix comprises a trivalent metal. This abstract is intended as a scanning tool for purposes of searching in the particular art and is not intended to be limiting of the present disclosure.

### CROSS-REFERENCE TO RELATED APPLICATIONS

This application claims the benefit of and priority to U.S. Provisional Application No. 63/752,942 filed on Feb. 3, 2025, which is incorporated herein by reference in its entirety.

### GOVERNMENT RIGHTS STATEMENT

The present invention was made with government support under contract number DE-SC0012704 awarded by the U.S. Department of Energy. The United States government may have certain rights in this invention.

### BACKGROUND

Current industrial methods for propene epoxidation primarily rely on the use of hydrogen peroxide as the oxidant. This introduces operational and environmental hurdles due to the corrosive nature of hydrogen peroxide. While silver-based catalysts are used in the industry for ethene and butene epoxidation with molecular oxygen, extensive academic and industrial research has shown that their performance for propene epoxidation is limited. Copper-based oxide catalysts have shown promise for partial oxidation reactions with molecular oxygen, but they rapidly and permanently deactivate under the required oxidizing conditions. Despite advances in strategies for the production of oxidized hydrocarbons, such as propene oxide, there remains a need for methods and catalysts for a more efficient, cost-effective, and safer production process. These needs and other needs are satisfied by the present disclosure.

### SUMMARY

In accordance with the purpose(s) of the present disclosure, as embodied and broadly described herein, the disclosure, in one aspect, relates to methods and compositions for the oxidation of hydrocarbons. In one aspect, disclosed is a method comprising: flowing a first gas mixture over a catalyst, wherein the first gas mixture comprises a reducing agent; and flowing a second gas mixture comprising a hydrocarbon over the catalyst, thereby producing a gaseous product comprising an oxidized hydrocarbon; wherein the catalyst comprises Cuⁿ⁺ ions dispersed in an oxide matrix, where n is an integer ranging from 0 to 2; and wherein the oxide matrix comprises a trivalent metal.

Other systems, methods, features, and advantages of the present disclosure will be or become apparent to one with skill in the art upon examination of the following drawings and detailed description. It is intended that all such additional systems, methods, features, and advantages be included within this description, be within the scope of the present disclosure, and be protected by the accompanying claims. In addition, all optional and preferred features and modifications of the described embodiments are usable in all aspects of the disclosure taught herein. Furthermore, the individual features of the dependent claims, as well as all optional and preferred features and modifications of the described embodiments are combinable and interchangeable with one another.

### BRIEF DESCRIPTION OF THE DRAWINGS

Many aspects of the present disclosure can be better understood with reference to the following drawings. The components in the drawings are not necessarily to scale, emphasis instead being placed upon clearly illustrating the principles of the present disclosure. Moreover, in the drawings, like reference numerals designate corresponding parts throughout the several views.

FIG. 1 shows a flowchart illustrating one example of a disclosed method for catalyzing hydrocarbon to an oxidized hydrocarbon in the presence of molecular oxygen.

FIG. 2 shows a schematic illustration of a representative catalytic testing setup for propene conversion to propene oxide.

FIG. 3 shows a representative process outline for propene conversion to propene oxide using a gallium catalyst (CuGaO₂).

FIG. 4A shows percent conversion of propene (PP) for a representative conversion process of propene to propene oxide using a CuGaO₂ catalyst.

FIG. 4B shows selectivities for CO₂, acrolein (C₃H₄O), and propene oxide (C₃H₆O) for a representative conversion process of propene to propene oxide using a CuGaO₂ catalyst.

FIGS. 5A and 5B show a representative chromatogram for propene oxide (PO) production at 175° C. using a CuGaO₂ catalyst (FIG. 5A) and a close-up view of the identification zone of PO and acrolein (FIG. 5B).

FIGS. 6A and 6B show a representative chromatogram of PO calibration (FIG. 6A) and a close-up view of the identification zone of PO (FIG. 6B).

FIGS. 7A and 7B show a representative chromatogram of acrolein calibration (FIG. 7A) and a close-up view of the identification zone of acrolein (FIG. 7B).

FIG. 8A shows a schematic illustration of a representative catalytic testing setup for propene conversion to propene oxide using an iron catalyst (CuFeO₂).

FIGS. 8B-8D show images depicting various components of the catalytic testing setup of FIG. 8A.

FIGS. 9A and 9B show X-ray diffraction (XRD) spectra of a representative CuFeO₂ catalyst along with reference spectra JCPDS 39-0246 (bottom of FIG. 9B).

FIG. 10 shows a Brunauer-Emmett-Teller (BET) adsorption/desorption graph for a representative CuFeO₂ catalyst.

FIG. 11 shows a representative process outline for propene conversion to propene oxide using an iron catalyst (CuFeO₂).

FIG. 12A shows percent conversion of propene (PP) for a representative conversion process of propene to propene oxide at various temperatures using a CuFeO₂ catalyst and Condition 1.

FIG. 12B shows percent yield of propene oxide (PO) for a representative conversion process of propene to propene oxide at various temperatures using a CuFeO₂ catalyst and Condition 1.

FIGS. 13A-13C shows selectivities for CO₂, acrolein, propene oxide (PO), and acetald for a representative conversion process of propene to propene oxide using a CuFeO₂ catalyst, feed Condition 1, and a temperature of 250° C. (FIG. 13A), 200° C. (FIG. 13B), or 150° C. (FIG. 13C).

FIG. 14A shows percent conversion of propene (PP) for a representative conversion process of propene to propene oxide at various temperatures using a CuFeO₂ catalyst and Condition 2.

FIG. 14B shows percent yield of propene oxide (PO) for a representative conversion process of propene to propene oxide at various temperatures using a CuFeO₂ catalyst and Condition 2.

FIGS. 15A-15C shows selectivities for CO₂, acrolein, propene oxide (PO), and acetald for a representative conversion process of propene to propene oxide using a CuFeO₂ catalyst, feed Condition 2 (at steady state reaction conditions), and a temperature of 250° C. (FIG. 15A), 200° C. (FIG. 15B), or 150° C. (FIG. 15C).

FIG. 16 shows a representative process outline for propene conversion to propene oxide using a gallium catalyst (CuGaO₂).

FIG. 17 shows selectivities for CO₂, acrolein, propene oxide (PO), and acetald for a representative conversion process of propene to propene oxide using a CuGaO₂ catalyst and a temperature of 200° C.

FIG. 18A shows percent conversion of propene (PP) for a representative conversion process of propene to propene oxide using a quartz reactor or a stainless steel (SS) reactor and a CuFeO₂.

FIG. 18B shows percent yield of propene oxide (PO) for a representative conversion process of propene to propene oxide using a quartz reactor or a stainless steel (SS) reactor and a CuFeO₂.

FIGS. 18C and 18D show selectivities for CO₂, acrolein, propene oxide (PO), and acetald for a representative conversion process of propene to propene oxide using a CuFeO₂ catalyst and a quartz reactor (FIG. 18C) or a stainless steel (SS) reactor (FIG. 18D) at temperature of 175° C.

FIG. 19 shows a representative process outline for propene conversion to propene oxide using an iron catalyst (CuFeO₂).

FIG. 20 shows a representative chromatogram of PP conversion to PO at 250° C. using a CuFeO₂ catalyst.

Additional advantages of the invention will be set forth in part in the description which follows, and in part will be obvious from the description, or can be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### DETAILED DESCRIPTION

Many modifications and other embodiments disclosed herein will come to mind to one skilled in the art to which the disclosed compositions and methods pertain having the benefit of the teachings presented in the foregoing descriptions and the associated drawings. Therefore, it is to be understood that the disclosures are not to be limited to the specific embodiments disclosed and that modifications and other embodiments are intended to be included within the scope of the appended claims. The skilled artisan will recognize many variants and adaptations of the aspects described herein. These variants and adaptations are intended to be included in the teachings of this disclosure and to be encompassed by the claims herein.

Although specific terms are employed herein, they are used in a generic and descriptive sense only and not for purposes of limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure.

Any recited method can be carried out in the order of events recited or in any other order that is logically possible. That is, unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

While aspects of the present disclosure can be described and claimed in a particular statutory class, such as the system statutory class, this is for convenience only and one of skill in the art will understand that each aspect of the present disclosure can be described and claimed in any statutory class.

It is also to be understood that the terminology used herein is for the purpose of describing particular aspects only and is not intended to be limiting. Unless defined otherwise, all technical and scientific terms used herein have the same meaning as commonly understood by one of ordinary skill in the art to which the disclosed compositions and methods belong. It will be further understood that terms, such as those defined in commonly used dictionaries, should be interpreted as having a meaning that is consistent with their meaning in the context of the specification and relevant art and should not be interpreted in an idealized or overly formal sense unless expressly defined herein.

Prior to describing the various aspects of the present disclosure, the following definitions are provided and should be used unless otherwise indicated. Additional terms may be defined elsewhere in the present disclosure.

### A. Definitions

As used herein, “comprising” is to be interpreted as specifying the presence of the stated features, integers, steps, or components as referred to, but does not preclude the presence or addition of one or more features, integers, steps, or components, or groups thereof. Moreover, each of the terms “by”, “comprising,” “comprises”, “comprised of,” “including,” “includes,” “included,” “involving,” “involves,” “involved,” and “such as” are used in their open, non-limiting sense and may be used interchangeably. Further, the term “comprising” is intended to include examples and aspects encompassed by the terms “consisting essentially of” and “consisting of.” Similarly, the term “consisting essentially of” is intended to include examples encompassed by the term “consisting of.

As used in the specification and the appended claims, the singular forms “a,” “an” and “the” include plural referents unless the context clearly dictates otherwise. Thus, for example, reference to “a cation” or “a gas” includes, but is not limited to, two or more such cations, gases, and the like.

Reference to “a” chemical compound refers to one or more molecules of the chemical compound rather than being limited to a single molecule of the chemical compound. Furthermore, the one or more molecules may or may not be identical, so long as they fall under the category of the chemical compound. Thus, for example, “a” chemical compound is interpreted to include one or more molecules of the chemical, where the molecules may or may not be identical (e.g., different isotopic ratios, enantiomers, and the like).

It should be noted that ratios, concentrations, amounts, and other numerical data can be expressed herein in a range format. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it will be understood that the particular value forms a further aspect. For example, if the value “about 10” is disclosed, then “10” is also disclosed.

When a range is expressed, a further aspect includes from the one particular value and/or to the other particular value. For example, where the stated range includes one or both of the limits, ranges excluding either or both of those included limits are also included in the disclosure, e.g. the phrase “x to y” includes the range from ‘x’ to ‘y’ as well as the range greater than ‘x’ and less than ‘y’. The range can also be expressed as an upper limit, e.g. ‘about x, y, z, or less' and should be interpreted to include the specific ranges of ‘about x’, ‘about y’, and ‘about z’ as well as the ranges of ‘less than x’, less than y’, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y’, and ‘greater than z’. In addition, the phrase “about ‘x’ to ‘y’”, where ‘x’ and ‘y’ are numerical values, includes “about ‘x’ to about ‘y’”.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

As used herein, the terms “about,” “approximate,” “at or about,” and “substantially” mean that the amount or value in question can be the exact value or a value that provides equivalent results or effects as recited in the claims or taught herein. That is, it is understood that amounts, sizes, formulations, parameters, and other quantities and characteristics are not and need not be exact but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In such cases, it is generally understood, as used herein, that “about” and “at or about” mean the nominal value indicated ±10% variation unless otherwise indicated or inferred. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

As used herein, the terms “optional” or “optionally” means that the subsequently described event or circumstance can or cannot occur, and that the description includes instances where said event or circumstance occurs and instances where it does not.

The term “contacting” as used herein refers to bringing a disclosed analyte, compound, chemical, or material in proximity to another disclosed analyte, compound, chemical, or material as indicated by the context. In some instances, contacting can comprise both physical and chemical interactions between the indicated components. It is to be understood that chemical interactions can comprise a combination of covalent and non-covalent interactions, including one or more of ionic, dipolar, van der Waals interactions, and the like.

The term “hydrocarbon,” as used herein, denotes a compound consisting of hydrogen and carbon (i.e., unsubstituted). The compound may be straight-chain (i.e., unbranched), branched, or cyclic (including fused, bridging, and spirofused polycyclic) and may be completely saturated or may contain one or more units of unsaturation, but which is not aromatic. Unless otherwise specified, hydrocarbon refers to compounds containing 1-20 carbon atoms. Hydrocarbons include, but are not limited to, linear or branched, alkyl, alkenyl, and alkynyl groups, and hybrids thereof such as (cycloalkyl)alkyl, (cycloalkenyl)alkyl or (cycloalkyl)alkenyl.

The term “aldehyde” as used herein is represented by the formula A¹C(O)H, where A¹ can be hydrogen, alkyl, cycloalkyl, alkenyl, cycloalkenyl, alkynyl, cycloalkynyl, aryl, or a heteroaryl group as described herein.

The term “ketone” as used herein is represented by the formula A¹C(O)A², where A¹ and A² can be, independently, an alkyl, cycloalkyl, alkenyl, cycloalkenyl, alkynyl, cycloalkynyl, aryl, or heteroaryl group as described herein.

The term “ether” as used herein is represented by the formula A¹OA², where A¹ and A² can be, independently, an alkyl, cycloalkyl, alkenyl, cycloalkenyl, alkynyl, cycloalkynyl, aryl, or heteroaryl group described herein. The term ether can also encompass cyclic ethers such as epoxide.

The term “alkyl” as used herein is a branched or unbranched saturated hydrocarbon group of 1 to 24 carbon atoms, such as methyl, ethyl, n-propyl, isopropyl, n-butyl, isobutyl, s-butyl, t-butyl, n-pentyl, isopentyl, s-pentyl, neopentyl, hexyl, heptyl, octyl, nonyl, decyl, dodecyl, tetradecyl, hexadecyl, eicosyl, tetracosyl, and the like. The alkyl group can be cyclic or acyclic. The alkyl group can be branched or unbranched. The alkyl group can also be substituted or unsubstituted. For example, the alkyl group can be substituted with one or more groups including, but not limited to, alkyl, cycloalkyl, alkoxy, amino, ether, halide, hydroxy, nitro, silyl, sulfo-oxo, or thiol, as described herein. A “lower alkyl” group is an alkyl group containing from one to six (e.g., from one to four) carbon atoms. The term alkyl group can also be a C1 alkyl, C1-C2 alkyl, C1-C3 alkyl, C1-C4 alkyl, C1-C5 alkyl, C1-C6 alkyl, C1-C7 alkyl, C1-C8 alkyl, C1-C9 alkyl, C1-C10 alkyl, and the like up to and including a C1-C24 alkyl.

Throughout the specification “alkyl” is generally used to refer to both unsubstituted alkyl groups and substituted alkyl groups; however, substituted alkyl groups are also specifically referred to herein by identifying the specific substituent(s) on the alkyl group. For example, the term “halogenated alkyl” or “haloalkyl” specifically refers to an alkyl group that is substituted with one or more halide, e.g., fluorine, chlorine, bromine, or iodine. Alternatively, the term “monohaloalkyl” specifically refers to an alkyl group that is substituted with a single halide, e.g. fluorine, chlorine, bromine, or iodine. The term “polyhaloalkyl” specifically refers to an alkyl group that is independently substituted with two or more halides, i.e. each halide substituent need not be the same halide as another halide substituent, nor do the multiple instances of a halide substituent need to be on the same carbon. The term “alkoxyalkyl” specifically refers to an alkyl group that is substituted with one or more alkoxy groups, as described below. The term “aminoalkyl” specifically refers to an alkyl group that is substituted with one or more amino groups. The term “hydroxyalkyl” specifically refers to an alkyl group that is substituted with one or more hydroxy groups. When “alkyl” is used in one instance and a specific term such as “hydroxyalkyl” is used in another, it is not meant to imply that the term “alkyl” does not also refer to specific terms such as “hydroxyalkyl” and the like.

This practice is also used for other groups described herein. That is, while a term such as “cycloalkyl” refers to both unsubstituted and substituted cycloalkyl moieties, the substituted moieties can, in addition, be specifically identified herein; for example, a particular substituted cycloalkyl can be referred to as, e.g., an “alkylcycloalkyl.” Similarly, a substituted alkoxy can be specifically referred to as, e.g., a “halogenated alkoxy,” a particular substituted alkenyl can be, e.g., an “alkenylalcohol,” and the like. Again, the practice of using a general term, such as “cycloalkyl,” and a specific term, such as “alkylcycloalkyl,” is not meant to imply that the general term does not also include the specific term.

The term “cycloalkyl” as used herein is a non-aromatic carbon-based ring composed of at least three carbon atoms. Examples of cycloalkyl groups include, but are not limited to, cyclopropyl, cyclobutyl, cyclopentyl, cyclohexyl, norbornyl, and the like. The term “heterocycloalkyl” is a type of cycloalkyl group as defined above, and is included within the meaning of the term “cycloalkyl,” where at least one of the carbon atoms of the ring is replaced with a heteroatom such as, but not limited to, nitrogen, oxygen, sulfur, or phosphorus. The cycloalkyl group and heterocycloalkyl group can be substituted or unsubstituted. The cycloalkyl group and heterocycloalkyl group can be substituted with one or more groups including, but not limited to, alkyl, cycloalkyl, alkoxy, amino, ether, halide, hydroxy, nitro, silyl, sulfo-oxo, or thiol as described herein.

The term “alkenyl” as used herein is a hydrocarbon group of from 2 to 24 carbon atoms with a structural formula containing at least one carbon-carbon double bond. Asymmetric structures such as (A¹A²)C═C(A³A⁴) are intended to include both the E and Z isomers. This can be presumed in structural formulae herein wherein an asymmetric alkene is present, or it can be explicitly indicated by the bond symbol C═C. The alkenyl group can be substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol, as described herein.

The term “cycloalkenyl” as used herein is a non-aromatic carbon-based ring composed of at least three carbon atoms and containing at least one carbon-carbon double bound, i.e., C═C. Examples of cycloalkenyl groups include, but are not limited to, cyclopropenyl, cyclobutenyl, cyclopentenyl, cyclopentadienyl, cyclohexenyl, cyclohexadienyl, norbornenyl, and the like. The term “heterocycloalkenyl” is a type of cycloalkenyl group as defined above, and is included within the meaning of the term “cycloalkenyl,” where at least one of the carbon atoms of the ring is replaced with a heteroatom such as, but not limited to, nitrogen, oxygen, sulfur, or phosphorus. The cycloalkenyl group and heterocycloalkenyl group can be substituted or unsubstituted. The cycloalkenyl group and heterocycloalkenyl group can be substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol as described herein.

The term “alkynyl” as used herein is a hydrocarbon group of 2 to 24 carbon atoms with a structural formula containing at least one carbon-carbon triple bond. The alkynyl group can be unsubstituted or substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol, as described herein.

The term “cycloalkynyl” as used herein is a non-aromatic carbon-based ring composed of at least seven carbon atoms and containing at least one carbon-carbon triple bound. Examples of cycloalkynyl groups include, but are not limited to, cycloheptynyl, cyclooctynyl, cyclononynyl, and the like. The term “heterocycloalkynyl” is a type of cycloalkenyl group as defined above, and is included within the meaning of the term “cycloalkynyl,” where at least one of the carbon atoms of the ring is replaced with a heteroatom such as, but not limited to, nitrogen, oxygen, sulfur, or phosphorus. The cycloalkynyl group and heterocycloalkynyl group can be substituted or unsubstituted. The cycloalkynyl group and heterocycloalkynyl group can be substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol as described herein.

The term “aromatic group” as used herein refers to a ring structure having cyclic clouds of delocalized π r electrons above and below the plane of the molecule, where the π r clouds contain (4n+2) π r electrons. A further discussion of aromaticity is found in Morrison and Boyd, Organic Chemistry, (5th Ed., 1987), Chapter 13, entitled “Aromaticity,” pages 477-497, incorporated herein by reference. The term “aromatic group” is inclusive of both aryl and heteroaryl groups.

The term “aryl” as used herein is a group that contains any carbon-based aromatic group including, but not limited to, benzene, naphthalene, phenyl, biphenyl, anthracene, and the like. The aryl group can be substituted or unsubstituted. The aryl group can be substituted with one or more groups including, but not limited to, alkyl, cycloalkyl, alkoxy, alkenyl, cycloalkenyl, alkynyl, cycloalkynyl, aryl, heteroaryl, aldehyde, —NH₂, carboxylic acid, ester, ether, halide, hydroxy, ketone, azide, nitro, silyl, sulfo-oxo, or thiol as described herein. The term “biaryl” is a specific type of aryl group and is included in the definition of “aryl.” In addition, the aryl group can be a single ring structure or comprise multiple ring structures that are either fused ring structures or attached via one or more bridging groups such as a carbon-carbon bond. For example, biaryl to two aryl groups that are bound together via a fused ring structure, as in naphthalene, or are attached via one or more carbon-carbon bonds, as in biphenyl.

Unless otherwise specified, pressures referred to herein are based on atmospheric pressure (i.e. one atmosphere).

### B. Discussion

Propene oxide (PO) is an important intermediate in various chemical industries. For example, PO serves as an intermediate in the synthesis of various chemicals, including propylene glycol and polyether polyols, which have widespread applications ranging from aircraft de-icing fluids to food products. PO is currently produced in a relatively cost-ineffective and environment-unfriendly way. Direct propene epoxidation is proposed as the best alternative for current methods of PO production. However, there are a lack of efficient catalysts for this direct reaction, especially in relevant reaction conditions (high conversion).

Disclosed herein is a method for production of PO and other hydrocarbon oxides. In one aspect, the method uses a safer and more cost-effective oxidant—molecular oxygen. Also disclosed herein is a catalyst comprising stabilized Cu⁺ cations. In one aspect, the catalyst can be a copper-based oxide catalyst modified with a trivalent metal. Examples of trivalent metals include aluminum, chromium, iron, gallium, manganese, rhodium, iridium, cobalt, indium, yttrium, scandium, or a combination thereof. Without wishing to be bound by theory, in one aspect, the introduction of a trivalent metal in the catalyst can protect and maintain the active copper sites responsible for the oxidation reaction, preventing permanent catalyst deactivation. The disclosed methods offer safety and environmental benefits over existing methods, making it a potential tool for the large-scale production of propene oxide.

In one aspect, disclosed herein is a method comprising flowing a first gas mixture over a catalyst (e.g., contacting the catalyst with the first gas mixture), wherein the first gas mixture includes a reducing agent; and flowing a second gas mixture over the catalyst (e.g., contacting the catalyst with the second gas mixture), thereby producing a gaseous product. In one aspect, the second gas mixture comprises a hydrocarbon. In one aspect, the hydrocarbon can include a saturated hydrocarbon. In another aspect, the hydrocarbon can include an unsaturated hydrocarbon, including an unsubstituted alkenyl (e.g., propene), an unsubstituted cycloalkenyl, an unsubstituted alkynyl, an unsubstituted cycloalkynyl, or a combination thereof. In another aspect, the gaseous product comprises an oxidized hydrocarbon (e.g., ethers such as propene oxide). In one aspect, the catalyst can comprise Cuⁿ⁺ ions within an oxide matrix, where n represents the oxidation state of the copper ion and is an integer ranging from 0 to 2. The oxide matrix can comprise Cuⁿ⁺ ions and a trivalent metal such as aluminum, chromium, iron, gallium, manganese, rhodium, iridium, cobalt, indium, yttrium, scandium, or any combination thereof. The catalyst can be represented by CuMOₓ, where M is the trivalent metal. As used herein, CuMOₓ is used to represent a catalyst with a variety of atomic ratios depending on the charge of the copper ion (e.g., CuMO₂ for Cu⁺ or CuM₄O₃ for Cu⁰). An individual catalyst can comprise Cuⁿ⁺ ions in one or more oxidation states. The method can be carried out in a reactor, such as a fixed bed reactor. In one aspect, the catalyst can be a component of a catalyst bed and the catalyst bed can further comprise a solid diluent comprising an inert material. For example, the solid diluent can include a silica (e.g., quartz sand), alumina, silicon carbide, or a combination thereof. The catalyst bed can include the catalyst and the inert material in a weight ratio (catalyst:inert material) of about 1:10 to about 1:1, about 1:8 to about 1:1, about 1:5 to about 1:1, about 1:3 to about 1:1, about 1:2 to about 1:1, about 1:10 to about 4:5, about 1:10 to about 3:5, or about 1:5 to about 3:5. In one aspect, the catalyst bed includes a homogeneous or nearly homogenous mixture of the catalyst and the inert material. In another aspect, the catalyst bed includes one or more individual layers of the catalyst and the inert material.

In one aspect, the first gas mixture can include a reducing agent such as, for example, H₂, CO, a hydrocarbon, or a combination thereof. This hydrocarbon can be the same as or different from the hydrocarbon present in the second gas mixture. In a further aspect, the first gas mixtures can include from about 1% to about 20%, about 1% to about 15%, about 1% to about 10%, about 5% to about 20%, about 5% to about 15%, about 5% to about 10%, or about 10% to about 20% of the reducing agent by volume. In a further aspect, the first gas mixture can further include an inert gas, such as Ar, He, N₂, or a combination thereof. In one aspect, the inert gas can comprise the remaining volume of the first gas mixture. While the first gas mixture flows over the catalyst, the catalyst can be gradually heated to a temperature of about 100° C. to about 350° C., about 125° C. to about 350° C., about 150° C. to about 350° C., about 175° C. to about 350° C., about 200° C. to about 350° C., about 225° C. to about 350° C., about 250° C. to about 350° C., about 275° C. to about 350° C., about 100° C. to about 325° C., about 100° C. to about 300° C., about 100° C. to about 275° C., about 100° C. to about 250° C., about 100° C. to about 225° C., about 100° C. to about 200° C., about 100° C. to about 175° C., about 100° C. to about 150° C., about 200° C. to about 325° C., about 200° C. to about 300° C., about 200° C. to about 275° C., or about 250° C. to about 300° C. The catalyst can be slowly raised from the starting temperature of the process (e.g., room temperature) to the final temperature for a pre-determined period of time (e.g., from about 5 minutes to about 1 hour, about 15 minutes to about 1 hour, or about 5 minutes to about 45 minutes) or at a steady heating rate (e.g., about 2° C./min to about 50° C./min, about 2° C./min to about 25° C./min, or about 25° C./min to about 50° C./min). The catalyst and/or the reactor can be heated using a variety of techniques including, but not limited to, jacket heating, internal coil heating, electric heating (e.g., using an electric heating element in contact with the reactor), heating via infrared or microwave radiation, or electromagnetic induction heating.

In one aspect, in addition to the hydrocarbon, the second gas mixture can further comprise an inert gas (such as Ar, He, N₂, or a combination thereof), an oxidant (e.g., O₂), or a combination thereof. In another aspect, the second gas mixture can be introduced to the catalyst and/or the reactor along with a co-feed (e.g., contacting the catalyst with the co-feed). The second gas mixture and the co-feed introduction can be done at the same time or nearly the same time in the process (e.g., by beginning the flow of the second gas mixture and, a few seconds to a few minutes later, introducing the co-feed). The co-feed can include components such as water, CO₂, an alkyl chloride, or a combination thereof. In one aspect, the second gas mixture can include from about 70% to about 95%, about 75% to about 95%, about 80% to about 95%, or about 85% to about 95% of the inert gas by volume. In another aspect, the second gas mixture can include from about 1% to about 20%, about 1% to about 15%, about 1% to about 10%, about 5% to about 20%, about 5% to about 15%, about 5% to about 10%, or about 10% to about 20% of the hydrocarbon by volume. In another aspect, the second gas mixture can include the hydrocarbon and the O₂ in a hydrocarbon-to-O₂ volumetric ratio of about 1 to about 100, about 1 to about 75, about 1 to about 50, about 1 to about 25, about 1 to about 10, about 1 to about 5, about 5 to about 100, about 10 to about 100, about 25 to about 100, about 50 to about 100, or about 75 to about 100. In a further aspect, when the catalyst includes gallium as one of the or the only trivalent metal(s), the second gas mixture can include the hydrocarbon and the O₂ in a hydrocarbon-to-O₂ volumetric ratio of about 25 to about 150, about 50 to about 150, about 75 to about 150, or about 100 to about 150. In another further aspect, when the catalyst includes iron as one of the or the only trivalent metal(s), the second gas mixture can include the hydrocarbon and the O₂ in a hydrocarbon-to-O₂ volumetric ratio of about 2 to about 50, about 2 to about 25, or about 2 to about 10. In another aspect, the second gas mixture can include the hydrocarbon and the O₂ in a hydrocarbon-to-O₂ stoichiometric ratio of about 2 to about 100, about 2 to about 75, about 2 to about 50, about 2 to about 25, about 2 to about 10, about 25 to about 100, about 50 to about 100, about 75 to about 100, or about 25 to about 75. In a further aspect, when the catalyst includes gallium as one of the or the only trivalent metal(s), the second gas mixture can include the hydrocarbon and the O₂ in a hydrocarbon-to-O₂ stoichiometric ratio of about 25 to about 100, about 50 to about 100, about 75 to about 100, or about 25 to about 75. In another further aspect, when the catalyst includes iron as one of the or the only trivalent metal(s), the second gas mixture can include the hydrocarbon and the O₂ in a hydrocarbon-to-O₂ stoichiometric ratio of about 2 to about 50, about 2 to about 25, or about 2 to about 10.

The second gas mixture can be flowed over the catalyst at a rate of about 1 mol_{HC}/(mol_{Cu}·min) to about 25 mol_{HC}/(mol_{Cu}·min), about 1 mol_{HC}/(mol_{Cu}·min) to about 20 mol_{HC}/(mol_{Cu}·min), about 1 mol_{HC}/(mol_{Cu}·min) to about 15 mol_{HC}/(mol_{Cu}·min), about 1 mol_{HC}/(mol_{Cu}·min) to about 10 mol_{HC}/(mol_{Cu}·min), about 1 mol_{HC}/(mol_{Cu}·min) to about 5 mol_{HC}/(mol_{Cu}·min), about 5 mol_{HC}/(mol_{Cu}·min) to about 25 mol_{HC}/(mol_{Cu}·min), about 10 mol_{HC}/(mol_{Cu}·min) to about 25 mol_{HC}/(mol_{Cu}·min), about 15 mol_{HC}/(mol_{Cu}·min) to about 25 mol_{HC}/(mol_{Cu}·min), about 20 mol_{HC}/(mol_{Cu}·min) to about 25 mol_{HC}/(mol_{Cu}·min), about 5 mol_{HC}/(mol_{Cu}·min) to about 20 mol_{HC}/(mol_{Cu}·min), or about 10 mol_{HC}/(mol_{Cu}·min) to about 20 mol_{HC}/(mol_{Cu}·min); where mol_{HC} represents moles of the hydrocarbon in the second gas mixture and mol_{Cu} represents the moles of copper in the catalyst. The flow rate of the second gas mixture can be maintained throughout the step or can be adjusted (e.g., made faster or made slower) at one or more times during the step. In another aspect, prior to flowing the second gas mixture over the catalyst, an inert gas (e.g., N₂, Ar or He) can be flowed over the catalyst. In a further aspect, while the inert gas is flowing over the catalyst, the catalyst can be allowed to cool down to a temperature (e.g., room temperature) that is lower than its final, highest temperature that was reached while flowing the first gas mixture over the catalyst.

Prior to or during flowing the second gas mixture over the catalyst, the catalyst can be gradually heated to a temperature of about 50° C. to about 300° C., about 50° C. to about 275° C., about 50° C. to about 250° C., about 50° C. to about 200° C., about 50° C. to about 175° C., about 50° C. to about 150° C., about 75° C. to about 300° C., about 100° C. to about 300° C., about 125° C. to about 300° C., about 150° C. to about 300° C., about 175° C. to about 300° C., about 200° C. to about 300° C., about 200° C. to about 275° C., or about 200° C. to about 250° C. The catalyst can be slowly raised from whatever temperature it was at prior to this step (e.g., room temperature) to the final temperature for a pre-determined period of time or at a steady heating rate. The catalyst and/or the reactor can be heated using a variety of techniques including, but not limited to, jacket heating, internal coil heating, electric heating (e.g., using an electric heating element in contact with the reactor), heating via infrared or microwave radiation, or electromagnetic induction heating.

The catalyst can be a solid catalyst (e.g., a solid, particulate catalyst). In one aspect, the catalyst can include particles that have a shape described as spherical, ellipsoidal, cylindrical, planar, or any combination thereof. In one aspect, the catalyst includes particles with an average particle size of about 0.01 μm to about 100 μm, about 0.01 μm to about 50 μm, about 0.01 μm to about 10 μm, about 0.01 μm to about 1 μm, about 0.1 μm to about 100 μm, about 1 μm to about 100 μm, about 10 μm to about 100 μm, or about 0.1 μm to about 10 μm. The particle size can represent a measurement of a catalyst particle diameter. For non-spherical particles, the particle size can represent a measurement of the particle's longest diameter, diameter along the major axis, and/or longest diameter in regard to a cross sectional area of the particle. The catalyst can be further characterized by a Brunauer-Emmett-Teller (BET) surface area of about 1 m²/g to about 50 m²/g, about 1 m²/g to about 40 m²/g, about 1 m²/g to about 30 m²/g, about 1 m²/g to about 20 m²/g, about 1 m²/g to about 10 m²/g, about 5 m²/g to about 50 m²/g, about 5 m²/g to about 40 m²/g, about 5 m²/g to about 30 m²/g, about 5 m²/g to about 20 m²/g, or about 5 m²/g to about 10 m²/g. The BET surface area is a measurement of the total surface area of a sample, inclusive of any micropores (i.e., pores with a diameter of less than about 2 nm).

The gaseous product produced by the disclosed method can include a mixture of oxidized hydrocarbons and other compounds. For example, the gaseous product can include an epoxide (e.g., propene oxide), an aldehyde (e.g., acrolein), a ketone (e.g., acetone), an alcohol (e.g., propanol), an organic acid (e.g., acrylic acid), CO₂, CO, H₂O or a combination thereof. In one aspect, the catalyst can have a higher selectivity for the oxidized hydrocarbon than any other additional products that are produced via the disclosed method. In one aspect, the catalyst can have a selectivity for the oxidized hydrocarbon of about 30% to about 90%, about 30% to about 80%, about 30% to about 70%, about 30% to about 60%, about 40% to about 90%, about 50% to about 90%, about 60% to about 90%, about 40% to about 80%, about 40% to about 70%, about 50% to about 80%, or about 50% to about 70%. In another aspect, the method results in a hydrocarbon conversion of about 0.5% to about 20%, about 0.5% to about 15%, about 0.5% to about 10%, about 0.5% to about 5%, about 1% to about 20%, about 5% to about 20%, about 10% to about 20%, or about 1% to about 15%, or about 1% to about 10%. The hydrocarbon conversion value represents the percentage of hydrocarbon that, after contact with the catalyst, is oxidized (e.g., converted to an oxidized hydrocarbon) represents.

### C. Aspects

The following listing of exemplary aspects supports and is supported by the disclosure provided herein.

Aspect 1. A method comprising: flowing a first gas mixture over a catalyst, wherein the first gas mixture comprises a reducing agent; and flowing a second gas mixture comprising a hydrocarbon over the catalyst, thereby producing a gaseous product comprising an oxidized hydrocarbon; wherein the catalyst comprises Cuⁿ⁺ ions dispersed in an oxide matrix, where n is an integer ranging from 0 to 2; and wherein the oxide matrix comprises a trivalent metal.

Aspect 2. The method of aspect 1, wherein the reducing agent comprises H₂, CO, or a second hydrocarbon.

Aspect 3. The method of aspect 1 or aspect 2, wherein the hydrocarbon is an unsaturated hydrocarbon.

Aspect 4. The method of any one of aspects 1-3, wherein the hydrocarbon comprises propene.

Aspect 5. The method of any one of aspects 1-4, wherein flowing the first gas mixture further comprises heating the catalyst to a temperature of about 100° C. to about 350° C.

Aspect 6. The method of any one of aspects 1-5, wherein the second gas mixture comprises from about 1% to about 20% of the hydrocarbon by volume.

Aspect 7. The method of any one of aspects 1-6, wherein the second gas mixture comprises the hydrocarbon, Ar, He, N₂, O₂, or a combination thereof.

Aspect 8. The method of any one of aspects 1-7, wherein the second gas mixture comprises O₂ and the hydrocarbon in a hydrocarbon-to-O₂ volumetric ratio of about 1 to about 100.

Aspect 9. The method of any one of aspects 1-8, wherein flowing the second gas mixture over the catalyst further comprises contacting the catalyst with a co-feed comprising water, CO₂, an alkyl chloride, or a combination thereof.

Aspect 10. The method of any one of aspects 1-9, wherein flowing the second gas mixture further comprises heating the catalyst to a temperature of about 50° C. to about 300° C.

Aspect 11. The method of any one of aspects 1-10, further comprising flowing the second gas mixture over the catalyst at a rate of about 1 mol_{HC}/(mol_{Cu}·min) to about 25 mol_{HC}/(mol_{Cu}·min).

Aspect 12. The method of any one of aspects 1-11, further comprising flowing a third gas mixture over the catalyst prior to flowing the second gas mixture over the catalyst, wherein the third gas mixture comprises an inert gas.

Aspect 13. The method of any one of aspects 1-12, wherein the method is carried out in a reactor.

Aspect 14. The method of any one of aspects 1-13, wherein the catalyst is part of a catalyst bed, wherein the catalyst bed further comprises an inert material.

Aspect 15. The method of any one of aspects 1-14, wherein the catalyst bed comprises the catalyst and the inert material (IM) in a catalyst:IM weight ratio of about 1:10 to about 1:1.

Aspect 16. The method of any one of aspects 1-15, wherein the catalyst has a formula represented by CuMOₓ, wherein M represents the trivalent metal.

Aspect 17. The method of any one of aspects 1-16, wherein the catalyst has selectivity for the oxidized hydrocarbon of about 30% to about 90%.

Aspect 18. The method of any one of aspects 1-17, wherein about 0.5% to about 20% of the hydrocarbon is converted to the oxidized hydrocarbon.

Aspect 19. The method of any one of aspects 1-18, wherein the trivalent metal is selected from aluminum, chromium, iron, gallium, manganese, rhodium, iridium, cobalt, indium, yttrium, scandium, and any combination thereof.

Aspect 20. The method of any one of aspects 1-18, wherein the trivalent metal is selected from iron, gallium, and a combination thereof.

From the foregoing, it will be seen that aspects herein are well adapted to attain all the ends and objects hereinabove set forth together with other advantages which are obvious and which are inherent to the structure.

While specific elements and steps are discussed in connection to one another, it is understood that any element and/or steps provided herein is contemplated as being combinable with any other elements and/or steps regardless of explicit provision of the same while still being within the scope provided herein.

It will be understood that certain features and subcombinations are of utility and may be employed without reference to other features and subcombinations. This is contemplated by and is within the scope of the claims.

Since many possible aspects may be made without departing from the scope thereof, it is to be understood that all matter herein set forth or shown in the accompanying drawings and detailed description is to be interpreted as illustrative and not in a limiting sense.

It is also to be understood that the terminology used herein is for the purpose of describing particular aspects only, and is not intended to be limiting. The skilled artisan will recognize many variants and adaptations of the aspects described herein. These variants and adaptations are intended to be included in the teachings of this disclosure and to be encompassed by the claims herein.

Now having described the aspects of the present disclosure, in general, the following Examples describe some additional aspects of the present disclosure. While aspects of the present disclosure are described in connection with the following examples and the corresponding text and figures, there is no intent to limit aspects of the present disclosure to this description. On the contrary, the intent is to cover all alternatives, modifications, and equivalents included within the spirit and scope of the present disclosure.

### D. Examples

The following examples are put forth so as to provide those of ordinary skill in the art with a complete disclosure and description of how the compounds, compositions, articles, devices and/or methods claimed herein are made and evaluated, and are intended to be purely exemplary of the disclosure and are not intended to limit the scope of what the inventors regard as their disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### 1. Propene Epoxidation Using Gallium Catalysts

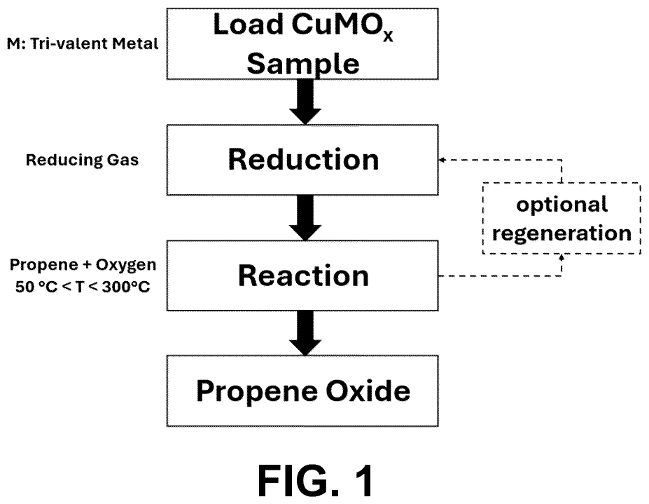

Discussed in this Example is the epoxidation of propene to propene oxide using molecular oxygen and a copper-based oxide catalyst. The process, detailed in FIG. 1, comprises the loading of a copper-based oxide catalyst modified by a trivalent metal into a catalytic reactor; activation of the catalyst by reduction with a reducing gas; reaction of propene with molecular oxygen; and collection of the resulting propene oxide.

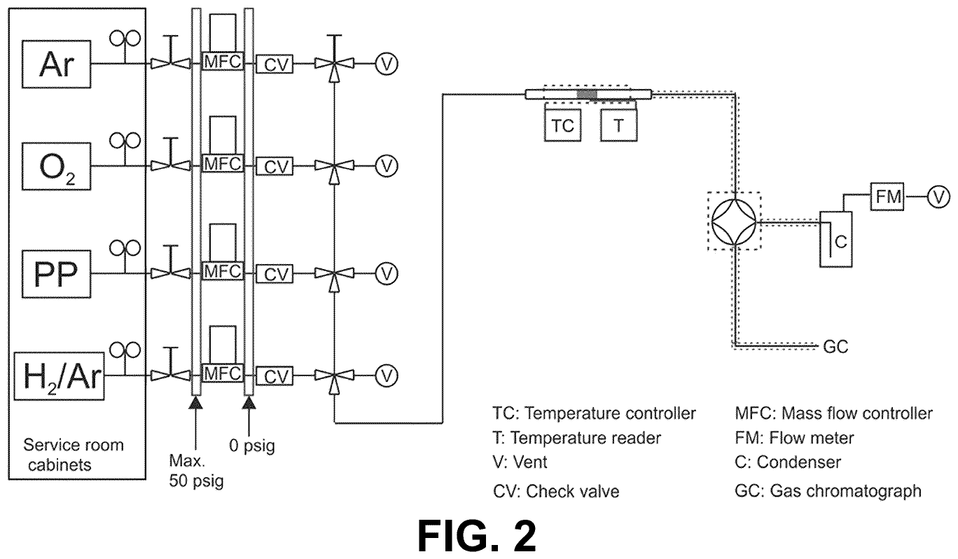

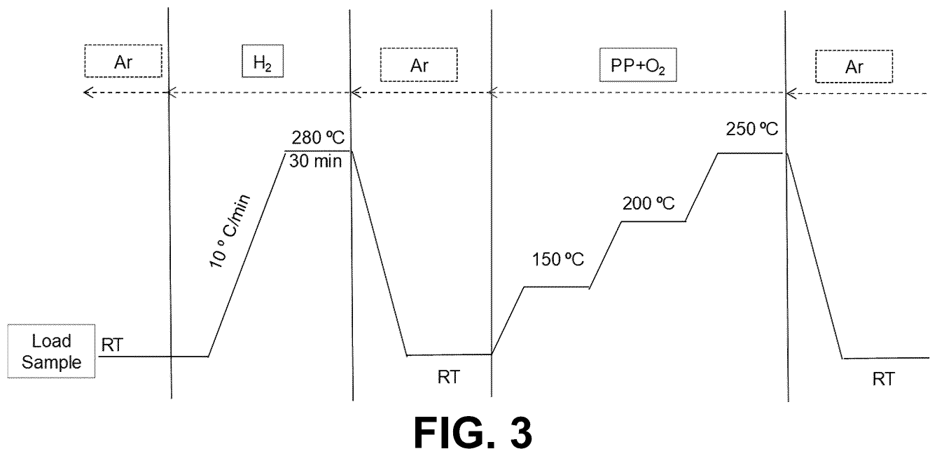

Different synthesis methods can be employed to prepare the catalyst containing stabilized Cu⁺ cations, and its performance can be further enhanced by catalytic modifiers. The specific reduction and reaction conditions, including gas and temperature for reduction step, reaction temperature (from 50° C. to 300° C.), pressure, and feed ratios, can be optimized to achieve optimum conversion and selectivity. A catalytic testing setup is shown in FIG. 2, where an inert gas (e.g. argon, He or N₂) was used as the carrier gas. An outline of the process for testing the gallium catalyst in this Example is depicted in FIG. 3. Pretreatment was done by flowing H₂ (10%) at 280° C. for 30 minutes (ramp at 10° C./min). The other process temperatures range from 100° C.-400° C. with a 50 cc/min total flow. Y_{PP}=8%, Y_{O2}=4%, 0.46 mol_{Cu}·s/mol_{PP}, 1 atm.

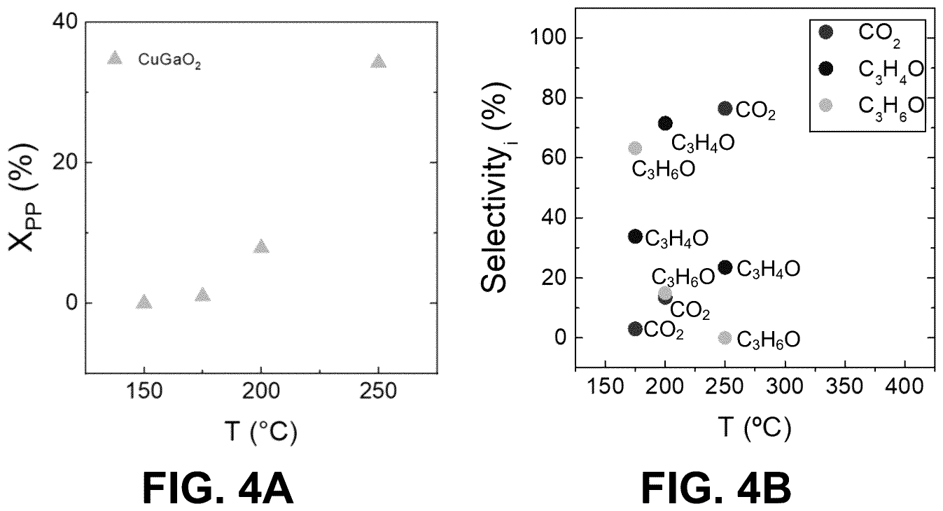

Example data is presented herein obtained using a copper-based oxide catalyst mixed with gallium as a trivalent metal (FIG. 4A and FIG. 4B). At a reaction temperature of 100° C., a selectivity of 100% towards propene oxide and a yield (C₃H₆O) of 0.1% is obtained, using a recirculation reactor with condensation of products. Advantages of the disclosed process and catalyst can include:
Enhanced Safety: The use of molecular oxygen as the oxidant eliminates the hazards and costs associated with hydrogen peroxide, leading to a safer process.
Improved Stability: The trivalent-metal modification significantly enhances the stability Cu⁺ active sites on copper-based oxide catalysts, extending their operational lifespan.
High Selectivity: The catalyst exhibits high selectivity for propene oxide formation, minimizing the formation of unwanted byproducts and improving process efficiency.
Cost-Effectiveness: The use of molecular oxygen, a readily available and inexpensive oxidant, reduces production costs compared to processes utilizing hydrogen peroxide.
Environmental Benefits: The direct epoxidation of propene with molecular oxygen offers a more environmentally friendly alternative to existing methods by reducing energy consumption and waste generation.
Propene Epoxidation Using CuMOₓ materials.

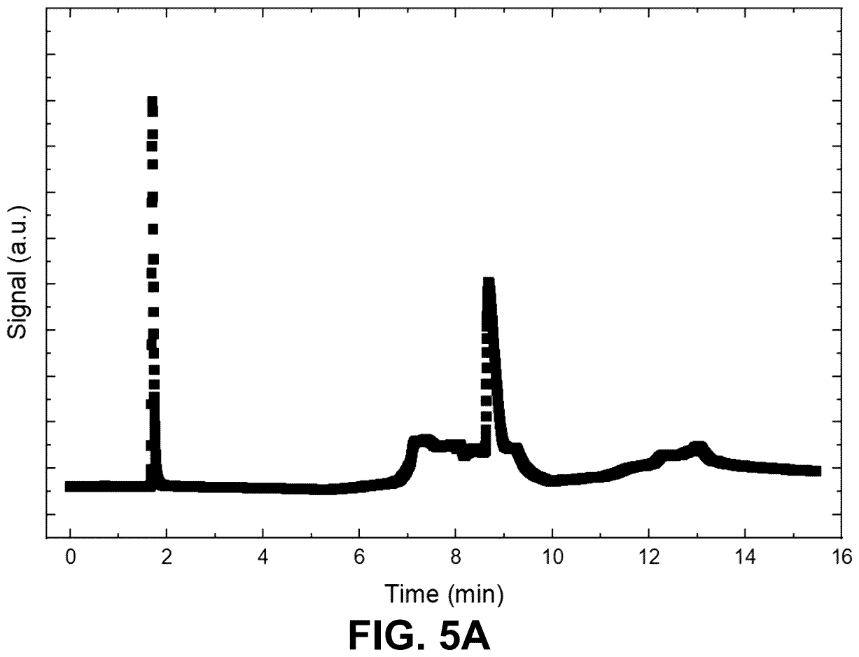

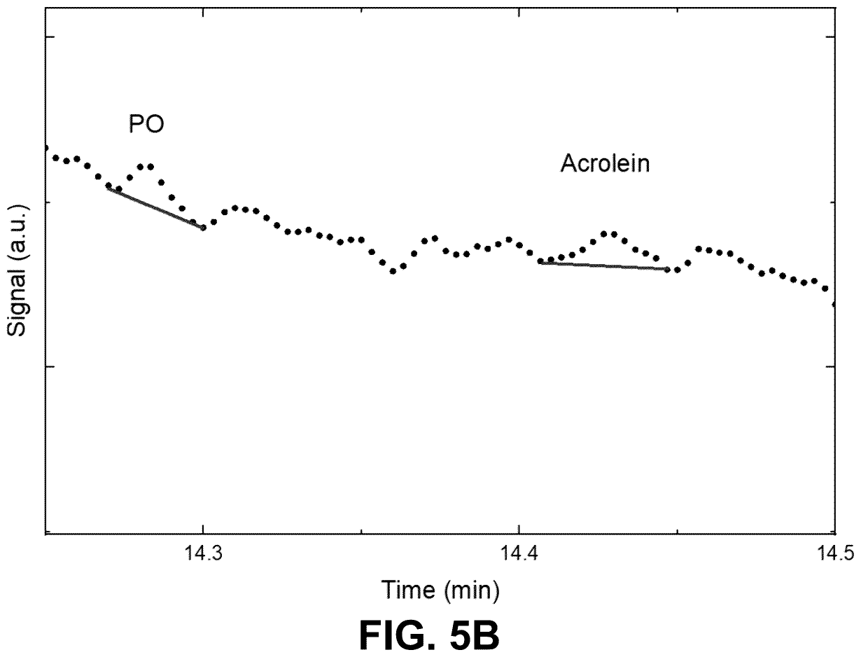

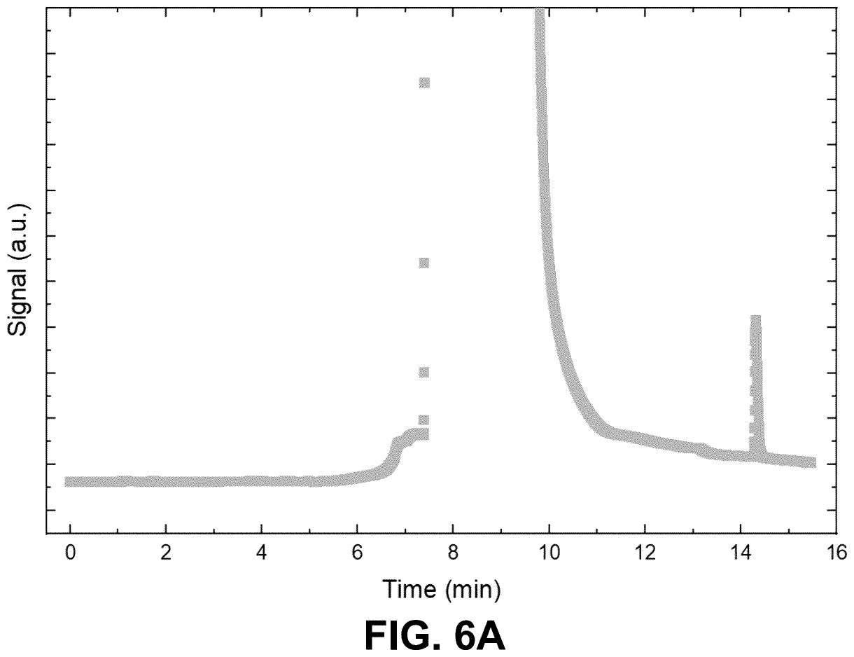

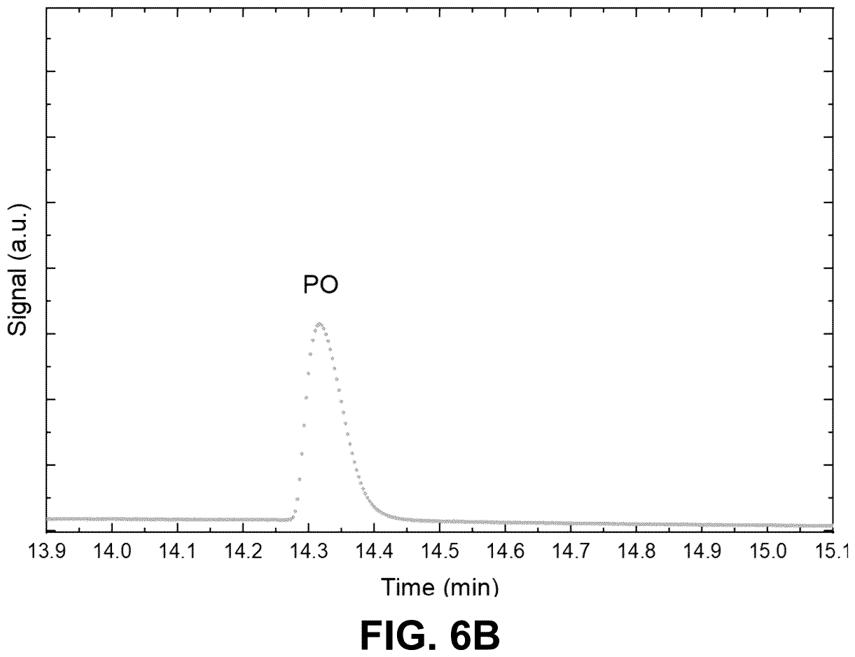

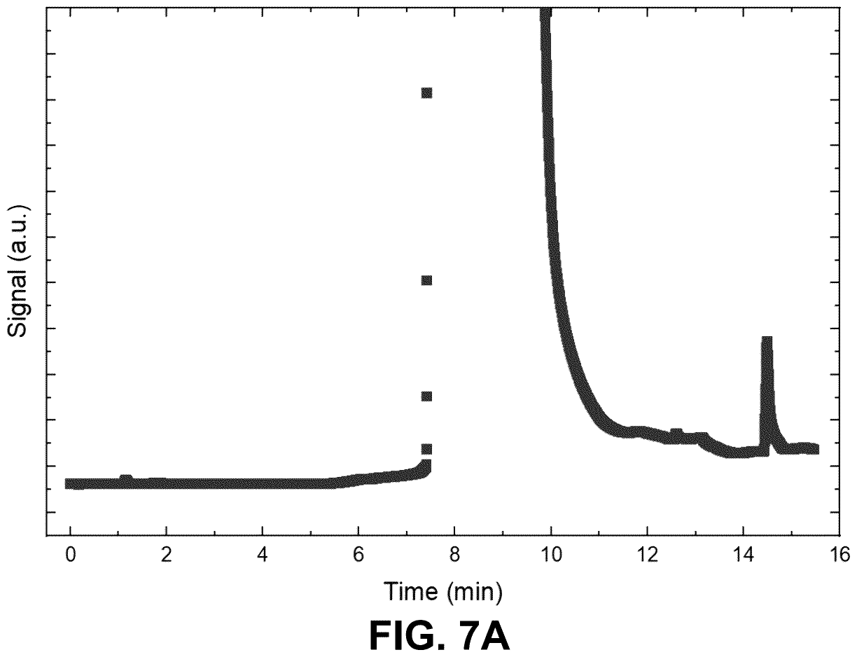

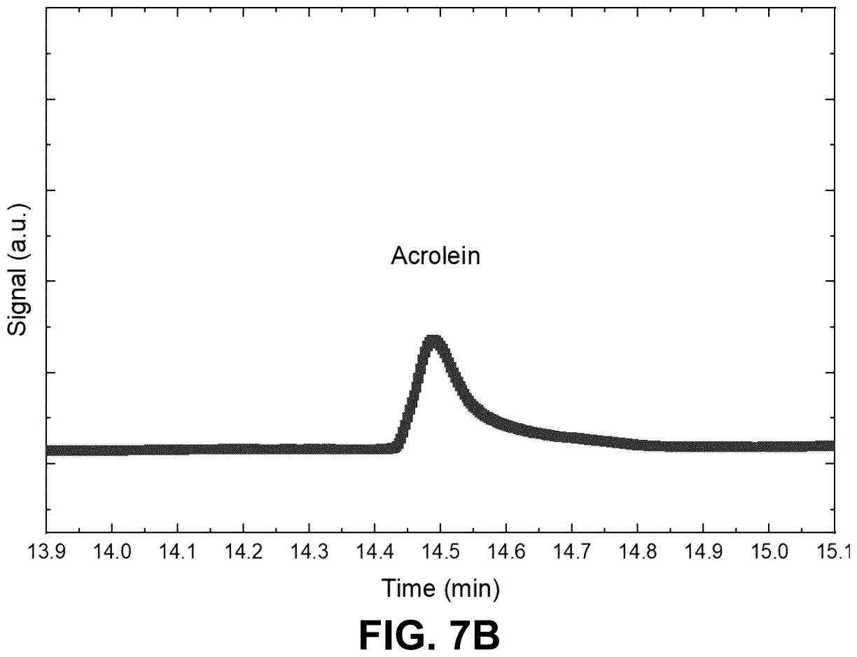

Chromatogram results are also provided for CuGaO₂. FIG. 5A shows the peaks of oxygen and propylene (the reactants) using argon as a carrier. At 7 min and between 12-13 min, signals from the baseline appear (they do not correspond to reactants or products). To identify the reaction products under differential conditions it is necessary to zoom in on the image (FIG. 5B). FIGS. 6A, 6B, 7A, and 7B show the chromatograms for propylene oxide and acrolein calibration.

Retention times for the reactants and products using argon as a carrier are provided in Table 1.

TABLE 1
Retention times for the reactants
and reaction products.

| Compound | Formulae | Retention time (min) |
|---|---|---|
| Oxygen | O₂ | 1.7 |
| Carbon dioxide | CO₂ | 2.4 |
| Ethylene | C₂H₄ | 3.3 |
| Water | H₂O | 8 |
| Propylene (PP) | C₃H₆ | 8.7 |
| Acetaldehyde | C₂H₄O | 12.5 |
| Propanal | C₃H₆O | 13.1-13.7 |
| Propylene oxide (PO) | C₃H₆O | 14.28 ± 0.04 |
| Acrolein | C₃H₄O | 14.44 ± 0.05 |
| Acetone | C₃H₆O | 14.52 ± 0.01 |

Reaction conditions at 175° C. were 25 cc/min, Y_{PP}=8%, Y_{O2}=4%, 50 mg of catalyst. Yield (PO) was 1.4%

The yield of 1.4% at a reaction temperature of 175° C. is higher than the best reported data in the literature of 0.9% for highly optimized potassium-promoted silver catalysts at a reaction temperature of 250° C.

At a reaction temperature of 100° C., a selectivity of 100% and a Yield (PO): 0.1% is obtained. At 100° C. a recirculation reactor is used with condensation of products (to avoid decomposition and accumulation of products).

### 2. Propene Epoxidation Using Gallium and Iron Catalysts

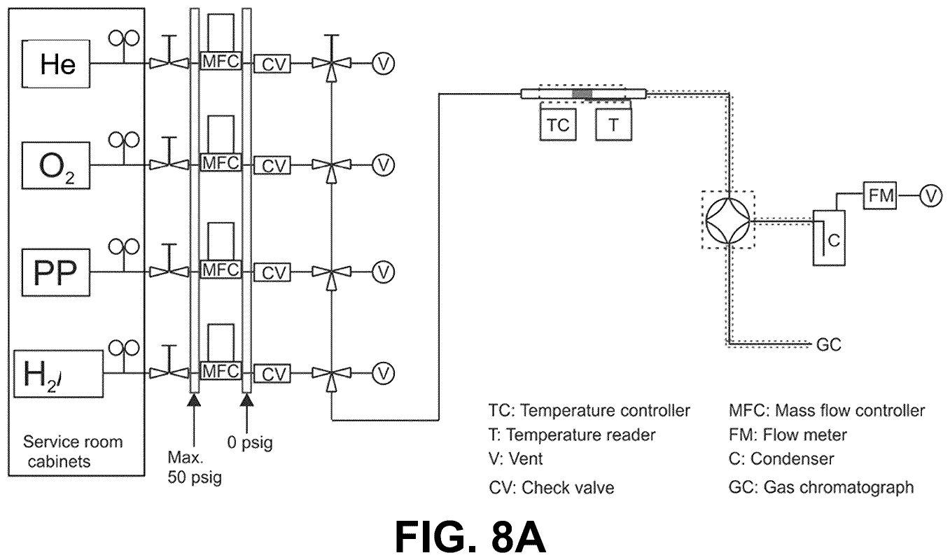

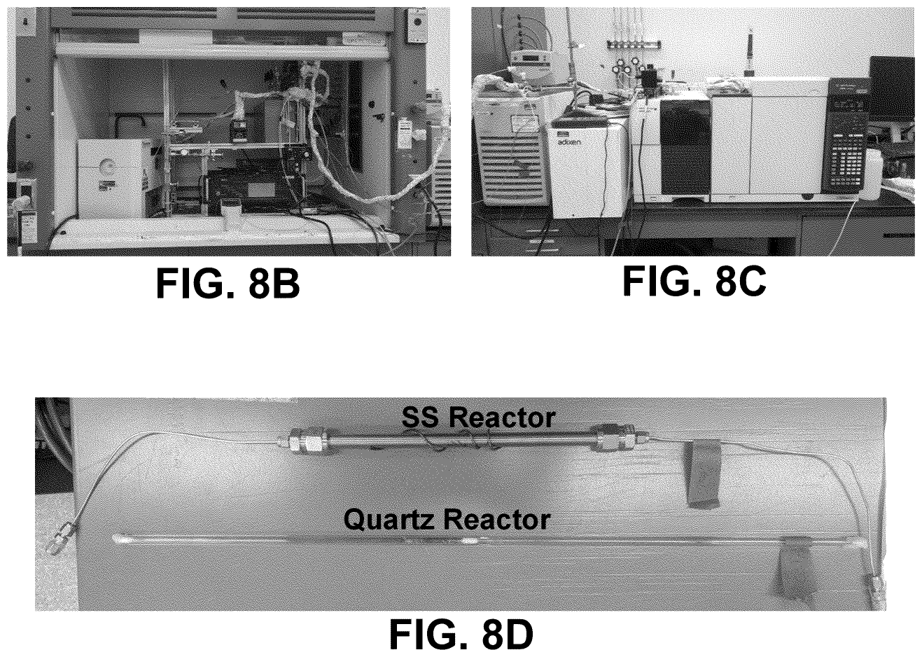

Discussed in this Example is the epoxidation of propene to propene oxide using molecular oxygen and a copper-based gallium oxide catalyst and iron oxide catalyst. A schematic catalytic testing setup is shown in FIG. 8A and pictures of the equipment shown in FIGS. 8B-8D, where argon or helium was used as the carrier gas for both the reaction system and the gas chromatography (GC). For the system, parameters for the stainless steel (SS) reactor are: Outer diameter (OD): ⅜ in (9.53 mm); Wall thickness: 0.049 in (1.24 mm); Inner diameter (ID): 0.277 in (7.04 mm); and Tube length: 8.27 in (210 mm). Parameters for the quartz reactor are: Outer diameter (OD): 0.25 in (6.35 mm); Wall thickness: ~0.060 in (1.52 mm); Inner diameter (ID): ~0.13 in (3.3 mm); and Tube length: 19.7 in (500 mm).

GC factors for the thermal conductivity detector using helium as a carrier gas and the GC conditions used are summarized in Table 2.

TABLE 2
Gas Chromatography factors and conditions used.

| Compound | Factor (umoles_{comp}/area) |
|---|---|
| CO₂ | 4.43E−04 |
| C₂H₄O | 3.93E−03 |
| C₃H₆O | 6.22E−04 |
| C₃H₄O | 3.95E−05 |

Selectivity was determined using Eq. (1):

$${S}_{i} = \frac{{m}_{i}^{out}}{x \cdot ( {m}_{{C}_{3} {H}_{6}}^{in} - {m}_{{C}_{3} {H}_{6}}^{out} )} \cdot 100 \qquad Eq . ( 1 )$$

In Eq. (1), m denotes the molar flow rate of reactants or products, in refers to the reactor inlet, out to the reactor outlet, i represents the product being analyzed, and x is the stoichiometric ratio between products and reactants.

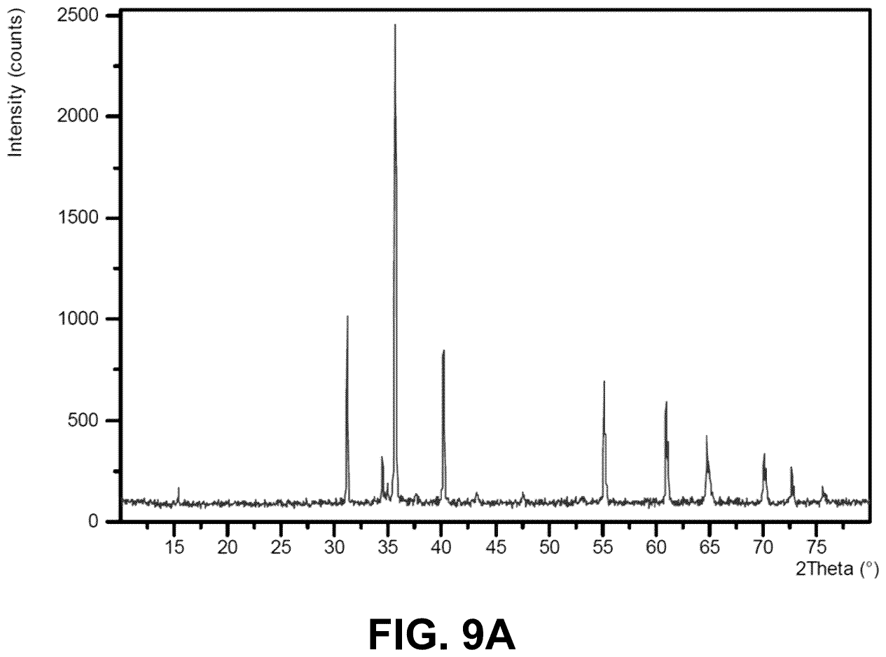

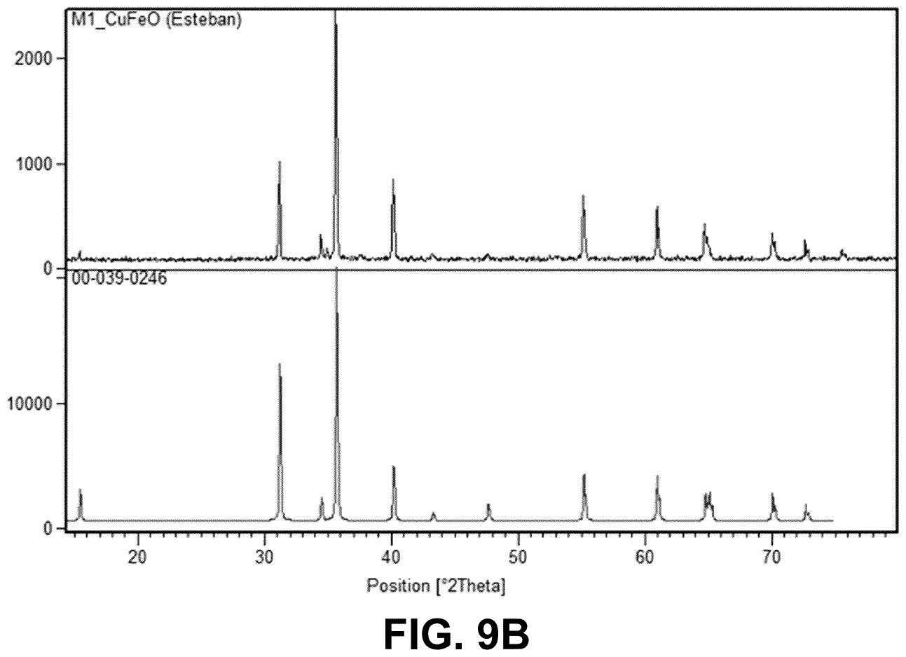

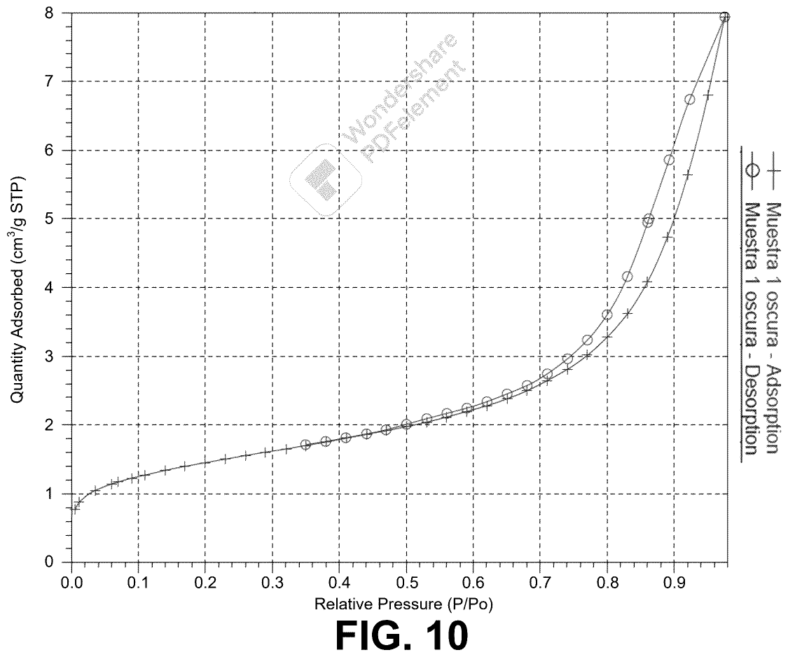

CuFeO₂ was synthesized using a microwave-assisted hydrothermal method. A stoichiometric mixture of Cu(NO₃)₂·3H₂O and Fe(NO₃)₃·9H₂O was introduced into a Teflon-lined vessel along with NaOH, glucose, and deionized water. The vessel was sealed and placed in a microwave reactor (Milestone Ethos UP), then heated to 200° C. for 30 min with a heating ramp of 7° C. min⁻¹. The resulting product was sequentially washed with diluted HNO₃ (1:1), aqueous ammonia, deionized water, and ethanol, followed by centrifugation and drying at 110° C. X-ray diffraction (XRD) spectra of CuFeO₂ is shown in FIGS. 9A and 9B. The XRD result shows the presence of a single phase corresponding to a pure phase of CuFeO₂ (reference spectra JCPDS 39-0246). The CuFeO₂ catalyst is further characterized in FIG. 10 by its BET surface area. The catalyst is characterized by a BET surface area of 4.6272 m²/g, a pore volume of 0.012281 cm³/g, an adsorption average pore width (4V/A by BET) of 106.1623 Å, and an average particle size of 12966.768 Å.

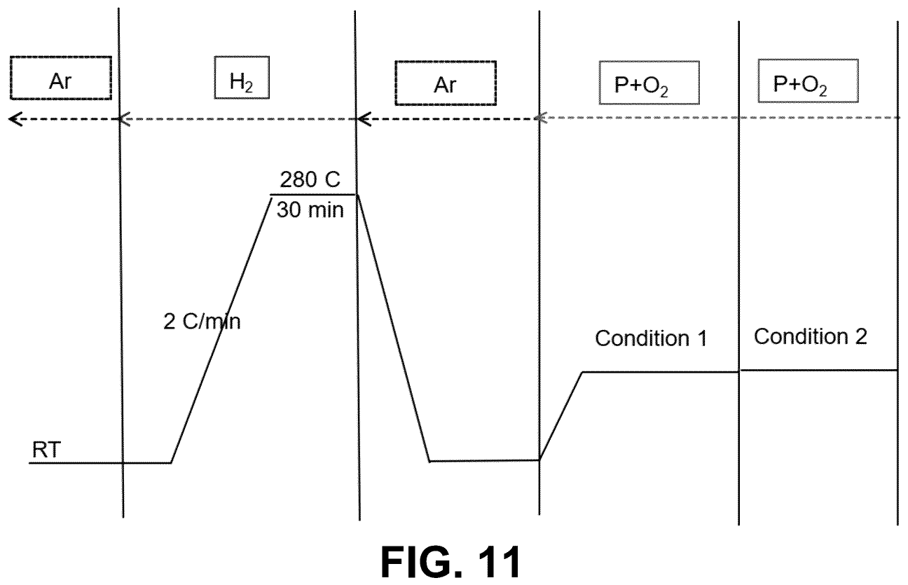

A CuFeO₂ catalyst was tested under the following conditions. An outline of the process is depicted in FIG. 11. Pretreatment was done by flowing H₂ (10%) at 280° C. for 30 minutes (ramp at 2° C./min). Two other testing conditions were used. Condition 1 is as follows: Total Flow: 13 cm³/min; Ar: 91.1%; O₂: 0.92; Propene (P): 8.00%; P/O₂-ratio: 8.7; (P/O₂)/(Stoichiometric-ratio): 4.3; weight of catalyst: 300 mg; Dilutant: 600 mg of quartz; and a quartz reactor. Condition 2 is as follows: Total Flow: 25 cm³/min; Ar: 91.1%; O₂: 0.92; P: 8.00%; P/O₂-ratio: 8.7; (P/O₂)/(Stoichiometric-ratio): 4.3; weight of catalyst: 300 mg; Dilutant: 600 mg of quartz; and a quartz reactor. The experiments were performed at 150° C., 200° C., 250° C., and 300° C.

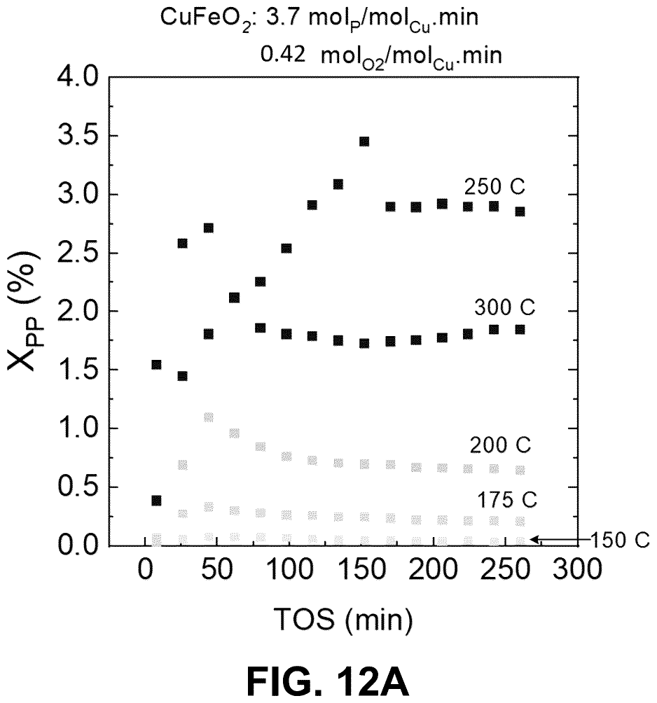

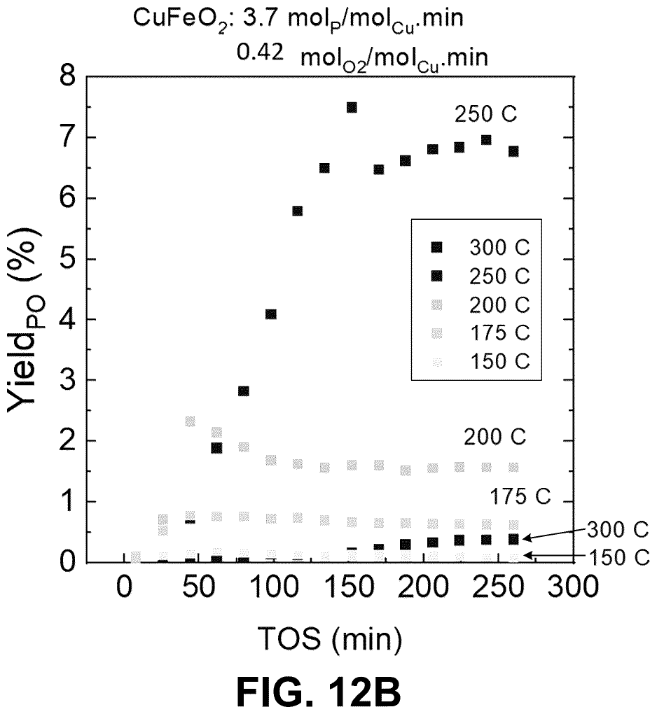

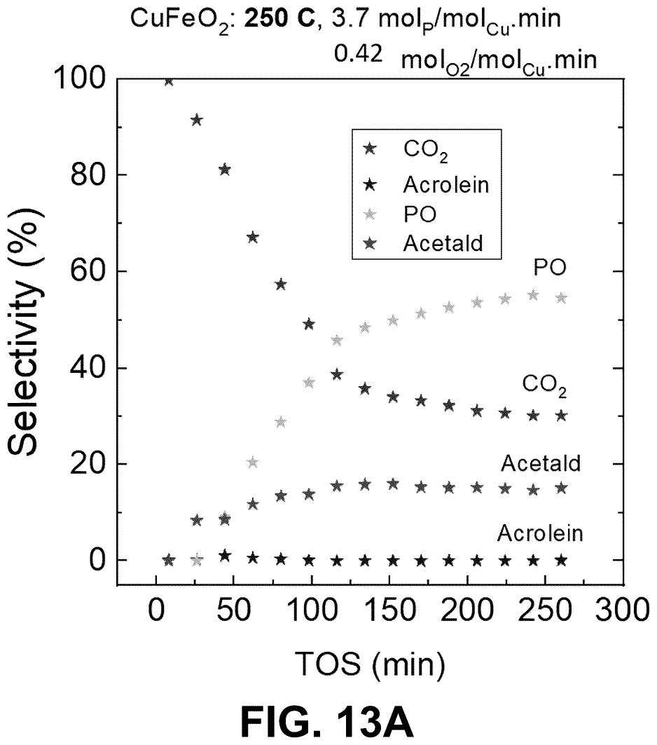

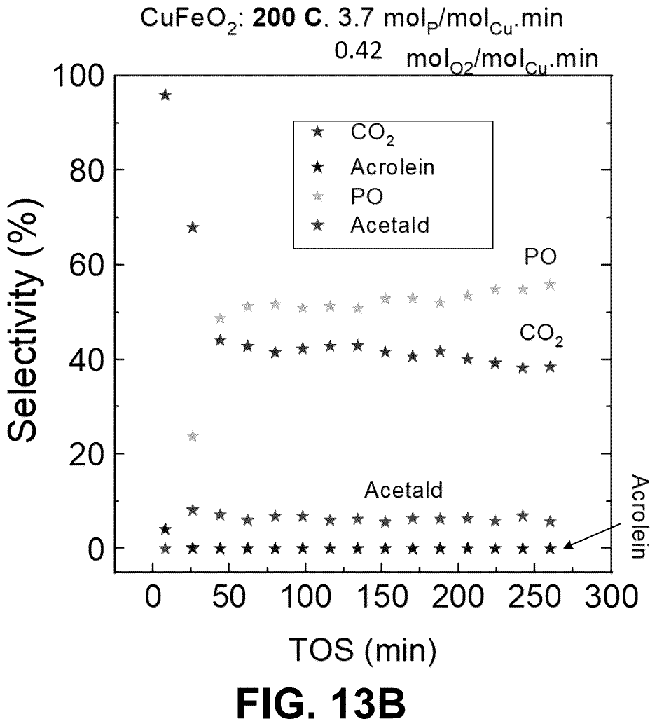

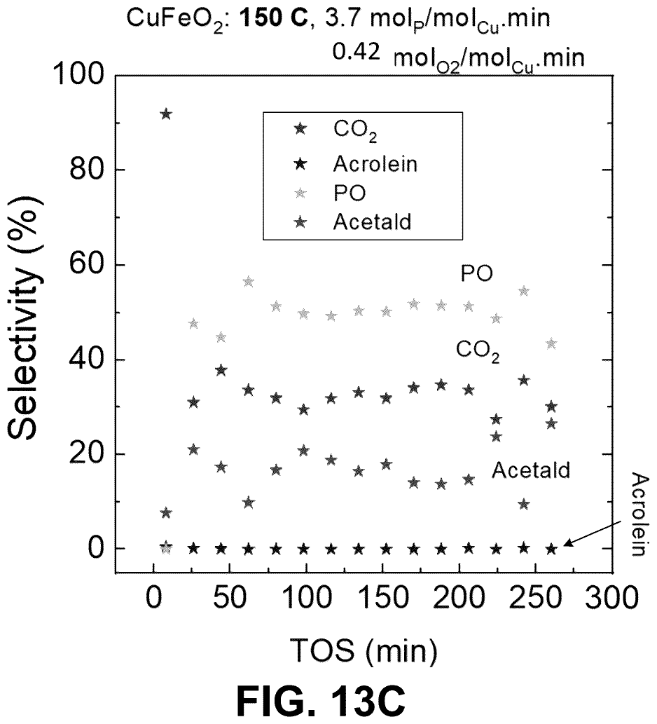

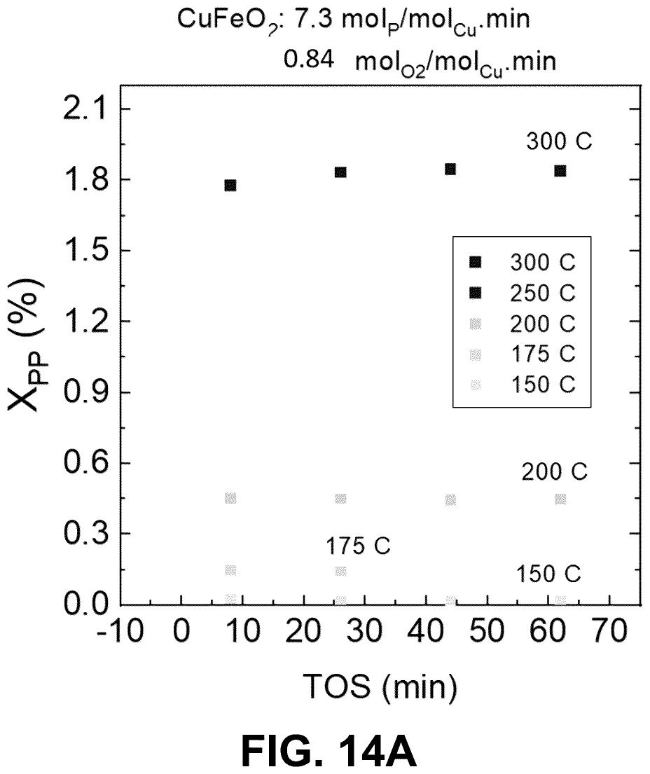

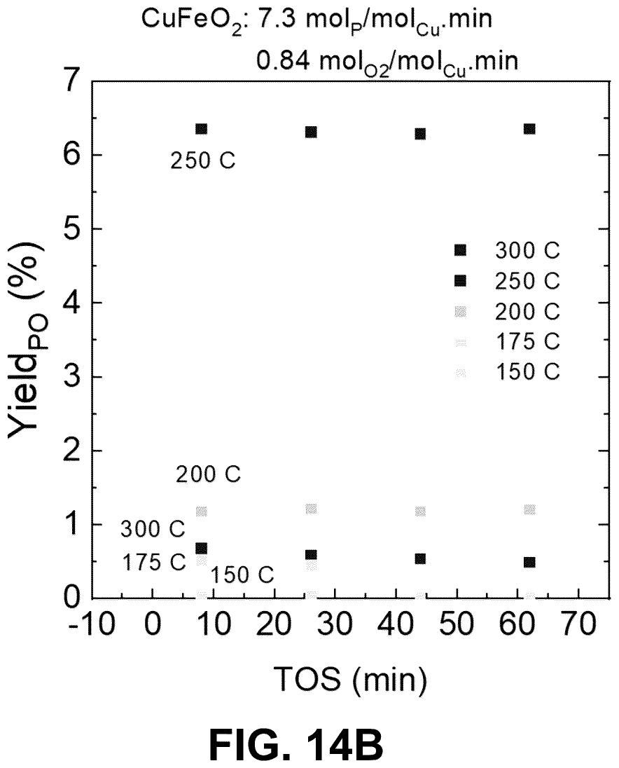

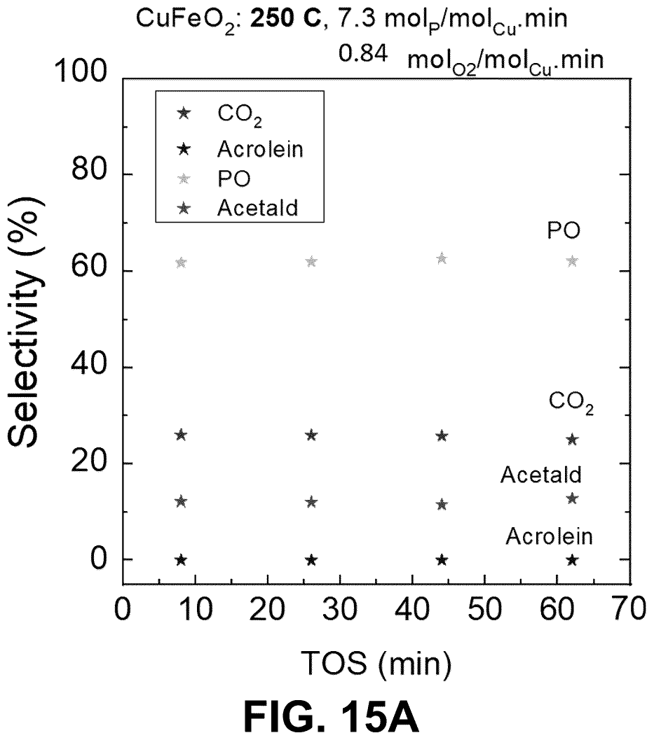

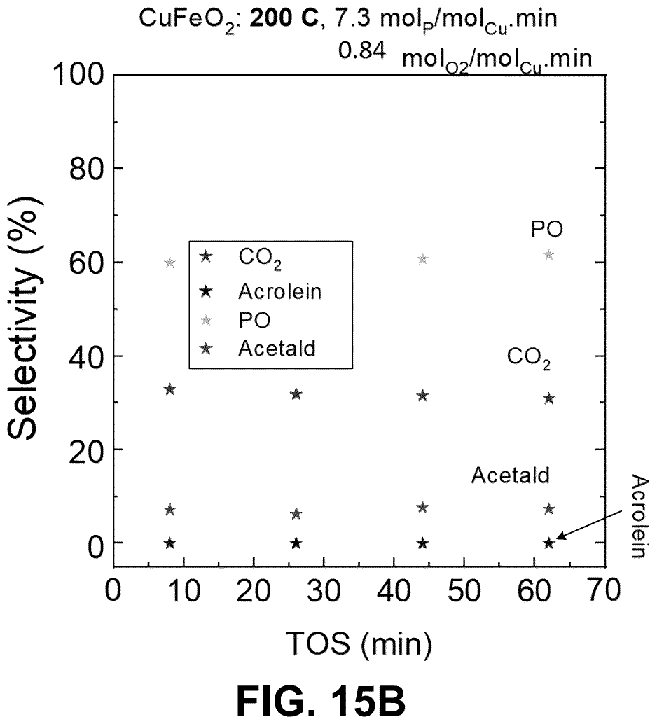

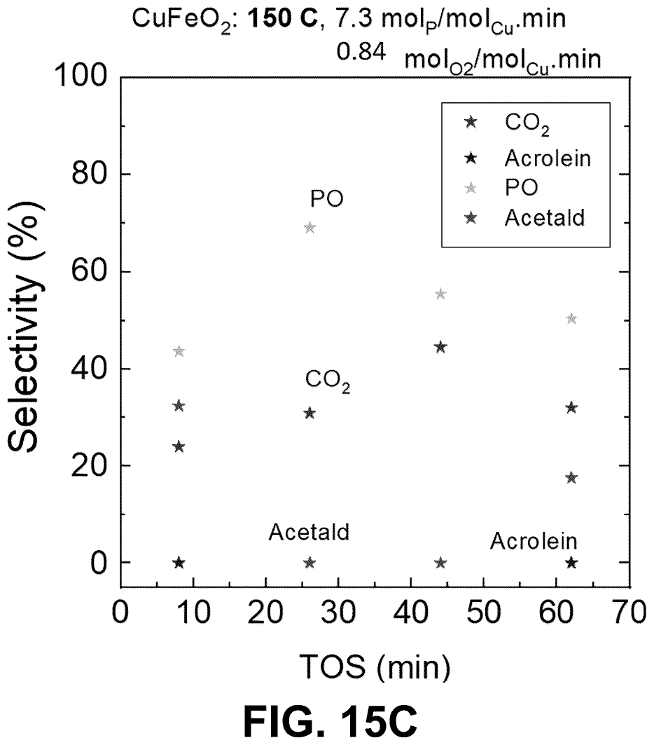

Results under Condition 1 are shown in FIGS. 12A and 12B. For FIG. 12A, a catalyst activation period was observed before reaching steady reaction conditions. Regarding FIG. 12B, the yield was determined based on oxygen. Selectivity results under Condition 1 are shown in FIGS. 13A-13C. Results under Condition 2 are shown in FIGS. 14A and 14B after the catalyst had reached steady state reaction conditions. Regarding FIG. 14B, the yield was determined based on oxygen. Selectivity results under Condition 2 are shown in FIGS. 15A-15C.

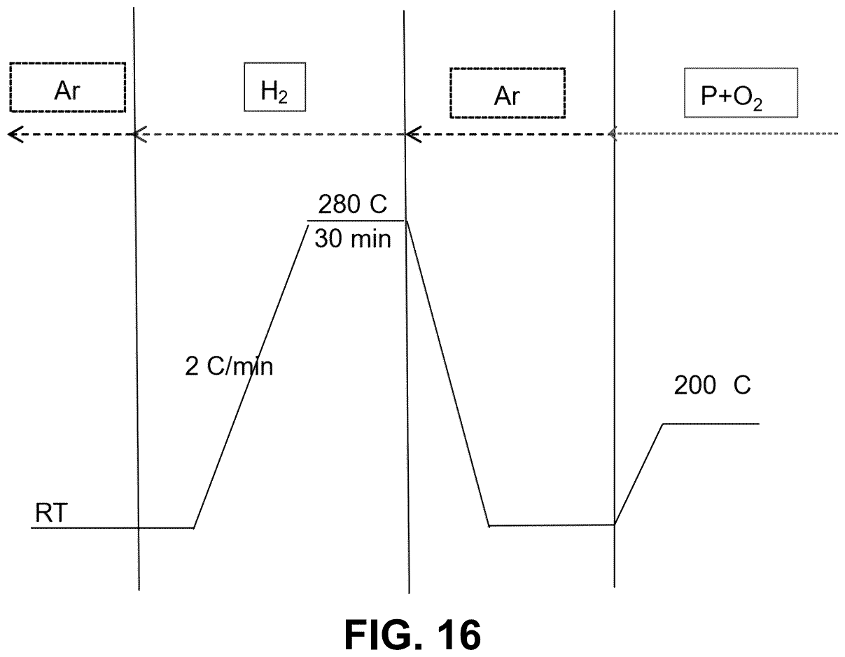

A CuGaO₂ catalyst was tested under conditions differing from Example 1. An outline of the process is depicted in FIG. 16. Pretreatment was done by flowing H₂ (10%) at 280° C. for 30 minutes (ramp at 2° C./min). The other conditions are as follows: Total Flow: 50 cm³/min; Ar: 83.84%; O₂: 0.16; P: 16.00%; P/O₂-ratio: 100; (P/O₂)/(Stoichiometric-ratio): 50; weight of catalyst: 70 mg; Dilutant: 140 mg of quartz; quartz reactor; and T: 200° C.

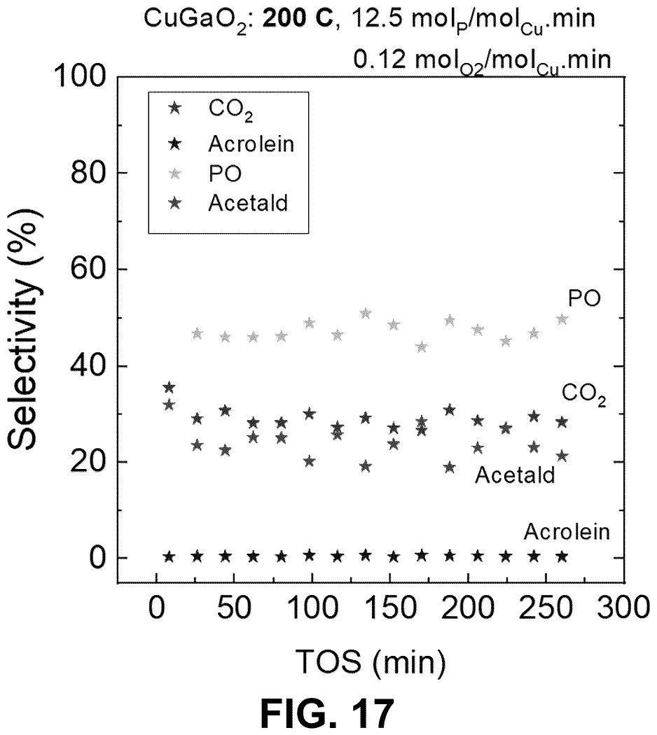

Adjusting the propylene/oxygen (p/O2) feed ratio to ~1/100 allows CuGaO₂ to improve selectivity reaching close to 50% at 200° C. (FIG. 17), from selectivities below 20% (FIG. 4B of Example 1) at the same temperature for stoichiometric feed ratios near ~1/2. This highlights the strong influence of the oxygen content in the feed on the catalytic performance. These conditions also lead to long time stable catalytic behavior, in contrast to the instability observed previously.

Performance of CuFeO₂ can be compared to CuGaO₂ via FIG. 17 and FIG. 13B. Regarding FIG. 13B, the P/O₂ ratio was 8.7. Regarding FIG. 17, the P/O₂ ratio was 100. In addition to improvement in PO selectivity for CuFeO₂ with respect to CuGaO₂ at the same reaction temperature, CuFeO₂ also remains stable even at higher O₂ feed ratios.

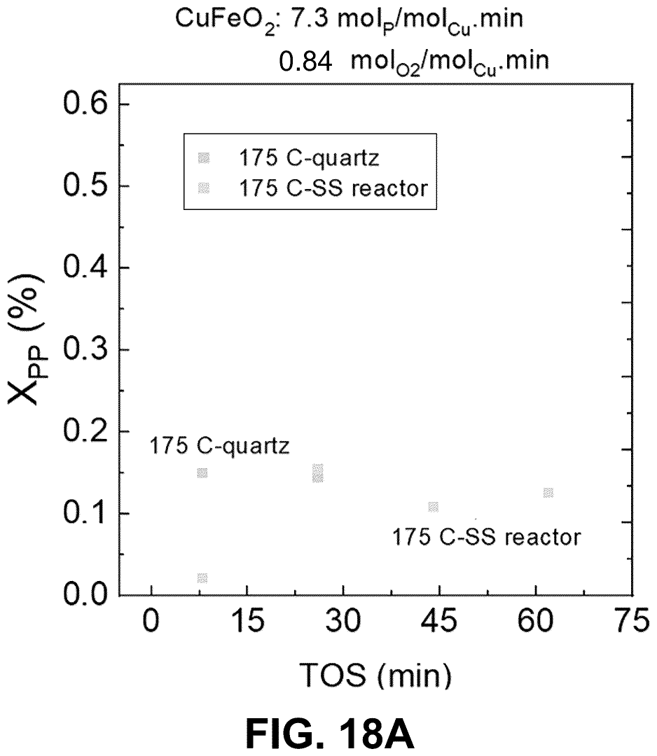

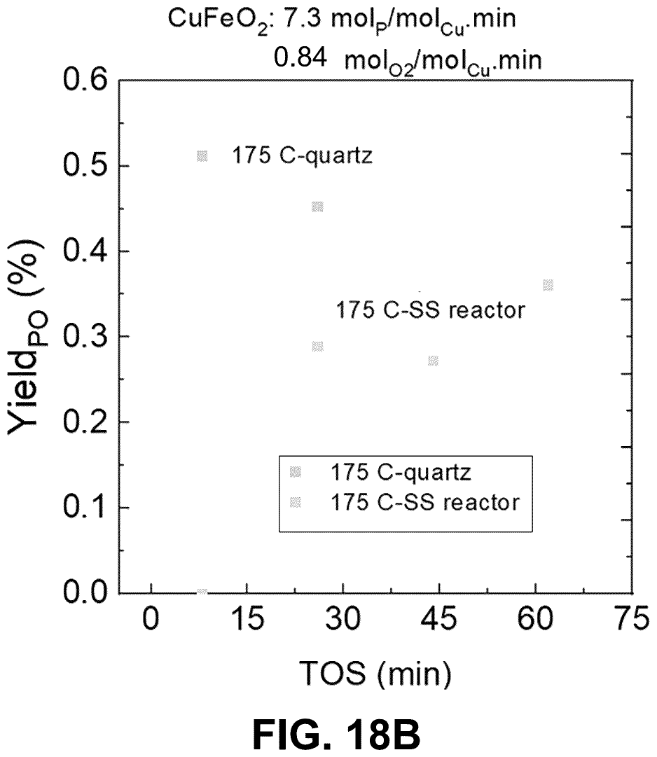

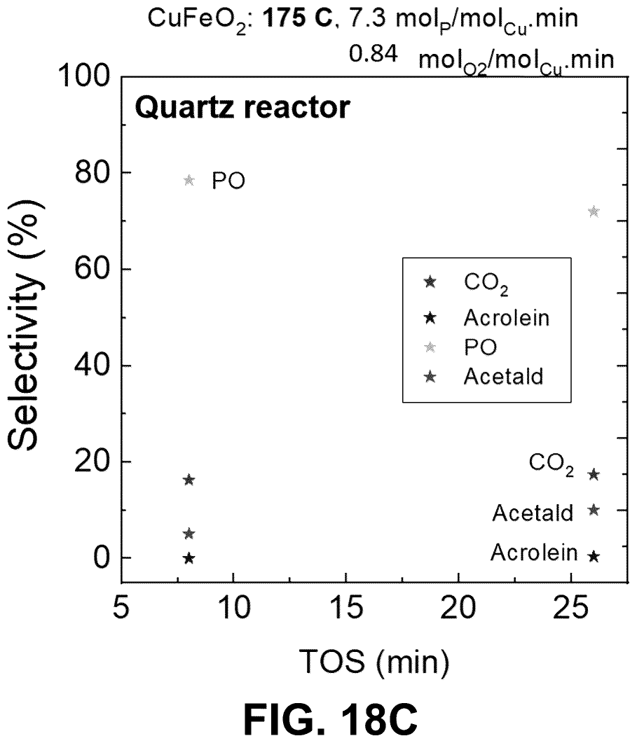

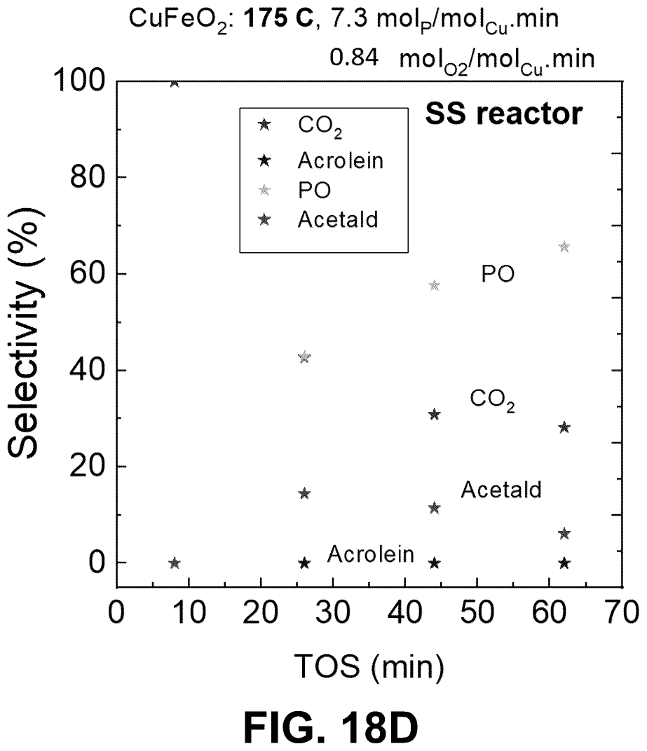

Additional results are shown for CuFeO₂ for a stainless steel reactor vs a quartz reactor (FIGS. 18A-18D), where the reactors converge to the same values over time.

Performance of CuFeO₂ was also evaluation with varying pretreatment conditions. Pretreatment 1 including flowing H₂ (10%) at 280° C. during 30 min (ramp at 2° C./min) and activation: Total Flow: 13 cm³/min; Ar: 91.1%; O₂: 0.92; P: 8.00%; P/O₂-ratio: 8.7; (P/O₂)/(Stoichiometric-ratio): 4.3; weight of catalyst: 300 mg; Dilutant: 600 mg of quartz; quartz reactor, T: 250° C.; and time: 3 h. Pretreatment 2 included flowing He at 250° C. during 30 min (ramp at 2° C./min) and additional conditions: Total Flow: 25 cm³/min; Ar: 91.1%; O₂: 0.92; P: 8.00%; P/O₂-ratio: 8.7; (P/O₂)/(Stoichiometric-ratio): 4.3; weight of catalyst: 300 mg; Dilutant: 600 mg of quartz; quartz reactor; and T: 250° C.

Results from pretreatment test are prestented in Table 3.

TABLE 3
Pretreatment test results.

| | Pretreatment 1 | Pretreatment 2 |
|---|---|---|
| Selectivity to PO (%) | 62 | 20 |
| Conversion Propylene (%) | 3 | 0.25 |

The reduction pretreatment aid in achieving meaningful propylene conversions and propylene oxide selectivities.

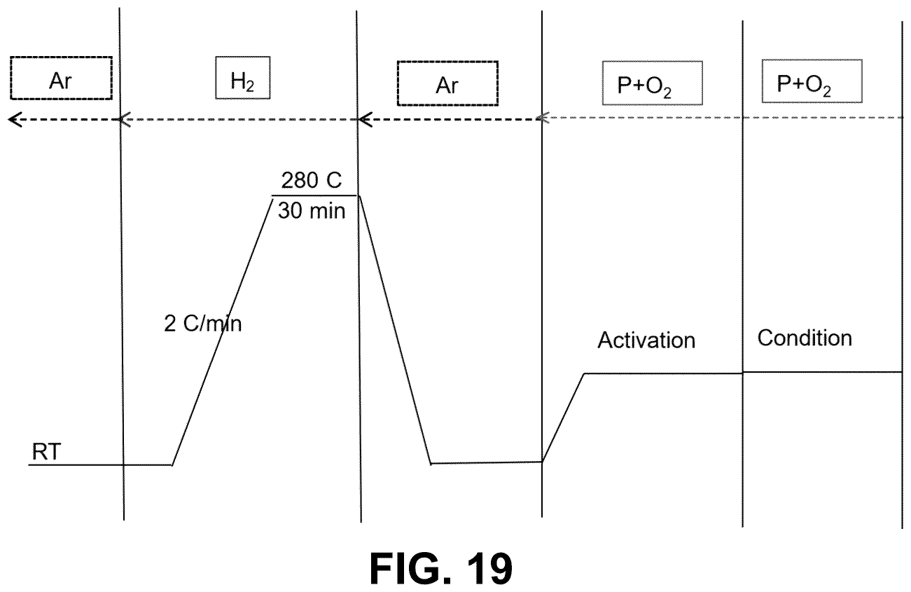

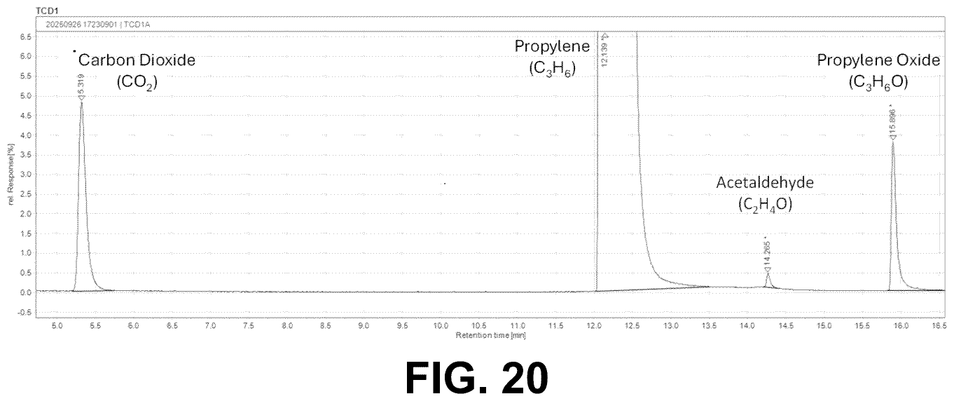

A summary of the best results obtained for CuFeO₂ are summarized in the process outline depicted in FIG. 19. Briefly, pretreatment included flowing H₂ (10%) at 280° C. during 30 min (ramp at 2° C./min). An activation process included: Total Flow: 13 cm³/min; Ar: 91.1%; O₂: 0.92; P: 8.00%; P/O₂-ratio: 8.7; (P/O₂)/(Stoichiometric-ratio): 4.3; weight of catalyst: 300 mg; Dilutant: 600 mg of quartz; quartz reactor, T: 250° C.; and time: 3 h. Additional Condition included: Total Flow: 25 cm³/min; Ar: 91.1%; O₂: 0.92; P: 8.00%; P/O₂-ratio: 8.7; (P/O₂)/(Stoichiometric-ratio): 4.3; weight of catalyst: 300 mg; Dilutant: 600 mg of quartz; quartz reactor; and T: 250° C. Results of chromatogram of experiments using helium as a carrier at 250° C. is shown in FIG. 20. Regarding FIG. 20, sensitivity factors vary with different chemicals and need to be calibrated by their proper reference.

Suggested targets for commercially viable catalysts for direct oxidation of propylene-to-propylene oxide (DOPO) using dioxygen (molecular oxygen) have been suggested in the patent literature. The suggested targets are based on early generation ethylene oxide (EO) processes with optimized catalysts, including promoters (e.g. carbonates, other transition metals) and co-feeds (e.g. water, alkyl chlorides or CO₂). In Table 4 reported values are from the disclosed catalyst without any promoters or co-feeds, and one case using a low concentration of water as co-feed.

TABLE 4
Propylene Oxide Catalytic Production Performance.

| | Suggested¹ | Disclosed | Disclosed |
|---|---|---|---|
| | Target | CuFeO₂ | CuFeO₂ |
| | (optimized | (at | (at 250 °C) |
| | process | 250 ° C.) | co-feed |
| | with co- | Over | water |
| | feeds and | 5 hours | p(H₂O)/ |
| | promoters) | of testing | p(C₃H₆) < 1 |
| Selectivity to PO (%) | 70 | 62 | 72 |
| Conversion Propylene (%) | 8 | 3 | 3 |
| Productivity [kg_{PO}/(m³_{cat}h)] | 150 | 31 | 34 |

¹based on literature review: Chojecki A., Ho C R., Sussman V J. Patent Literature on Epoxidation of Propylene Over Silver Catalysts Using Molecular Oxygen—A Critical Industrial Review. ChemCatChem 2024; 16(7). Doi: 10.1002/cctc.202301455.

In conclusion, suggested targets for commercially viable catalysts for direct oxidation of propylene-to-propylene oxide (DOPO) using dioxygen (molecular oxygen) are based on early generation ethylene oxide (EO) processes with optimized catalysts, including promoters and co-feeds. The values reported herein are using only the catalyst, without promoters or a low ratio of co-feeds which could improve selectivity (up to ~20%) and conversion (up to ~x5).

It should be emphasized that the above-described embodiments of the present disclosure are merely possible examples of implementations set forth for a clear understanding of the principles of the disclosure. Many variations and modifications may be made to the above-described embodiment(s) without departing substantially from the spirit and principles of the disclosure. All such modifications and variations are intended to be included herein within the scope of this disclosure and protected by the following claims.

What is claimed is:

1. A method comprising:
flowing a first gas mixture over a catalyst, wherein the first gas mixture comprises a reducing agent; and
flowing a second gas mixture comprising a hydrocarbon over the catalyst, thereby producing a gaseous product comprising an oxidized hydrocarbon;
wherein the catalyst comprises Cuⁿ⁺ ions dispersed in an oxide matrix, where n is an integer ranging from 0 to 2; and
wherein the oxide matrix comprises a trivalent metal.

2. The method of claim 1, wherein the reducing agent comprises H₂, CO, or a second hydrocarbon.

3. The method of claim 1, wherein the hydrocarbon is an unsaturated hydrocarbon.

4. The method of claim 1, wherein the hydrocarbon comprises propene.

5. The method of claim 1, wherein flowing the first gas mixture further comprises heating the catalyst to a temperature of about 100° C. to about 350° C.

6. The method of claim 1, wherein the second gas mixture comprises from about 1% to about 20% of the hydrocarbon by volume.

7. The method of claim 1, wherein the second gas mixture comprises the hydrocarbon, Ar, He, N₂, O₂, or a combination thereof.

8. The method of claim 1, wherein the second gas mixture comprises O₂ and the hydrocarbon in a hydrocarbon-to-O₂ volumetric ratio of about 1 to about 100.

9. The method of claim 1, wherein flowing the second gas mixture over the catalyst further comprises contacting the catalyst with a co-feed comprising water, CO₂, an alkyl chloride, or a combination thereof.

10. The method of claim 1, wherein flowing the second gas mixture further comprises heating the catalyst to a temperature of about 50° C. to about 300° C.

11. The method of claim 1, further comprising flowing the second gas mixture over the catalyst at a rate of about 1 mol_{HC}/(mol_{Cu}·min) to about 25 mol_{HC}/(mol_{Cu}·min).

12. The method of claim 1, further comprising flowing a third gas mixture over the catalyst prior to flowing the second gas mixture over the catalyst, wherein the third gas mixture comprises an inert gas.

13. The method of claim 1, wherein the method is carried out in a reactor.

14. The method of claim 1, wherein the catalyst is part of a catalyst bed, wherein the catalyst bed further comprises an inert material.

15. The method of claim 14, wherein the catalyst bed comprises the catalyst and the inert material (IM) in a catalyst:IM weight ratio of about 1:10 to about 1:1.

16. The method of claim 1, wherein the catalyst has a formula represented by CuMOₓ, wherein M represents the trivalent metal.

17. The method of claim 1, wherein the catalyst has selectivity for the oxidized hydrocarbon of about 30% to about 90%.

18. The method of claim 1, wherein about 0.5% to about 20% of the hydrocarbon is converted to the oxidized hydrocarbon.

19. The method of claim 1, wherein the trivalent metal is selected from aluminum, chromium, iron, gallium, manganese, rhodium, iridium, cobalt, indium, yttrium, scandium, and any combination thereof.

20. The method of claim 1, wherein the trivalent metal is selected from iron, gallium, and a combination thereof.
